(19) United States
(12) Patent Application Publication
(10) Pub. No.: US 2006/0246555 A1
(43) Pub. Date: Nov. 2, 2006

(54) Degradation of cyclic nitramines

(76) Inventor: Jalal Hawari, Montreal (CA)

Correspondence Address: BORDEN LADNER GERVAIS LLP, 1100-100 QUEEN ST, OTTAWA, ON, K1P 1J9, CA

(21) Appl. No.: 10/520,904

(22) PCT Filed: Nov. 27, 2002
(86) PCT No.: PCT/CA02/01799

§ 371 Date: Sep. 9, 2005

Related U.S. Application Data
(60) Provisional application No. 60/395,316, filed on Jul. 12, 2002.

(51) Int. Cl. A62D 3/00; C12P 13/00

(52) U.S. Cl. 435/128; 588/403; 588/306; 588/313

(57) ABSTRACT

The invention relates to a process for degradation of nitramines. The explosives RDX, HMX, CL20 and tetryl are exemplary nitramines that may be degraded according to the invention. These nitramines may be attacked chemically, biochemically or biologically. The invention may be used to degrade such explosive compounds that may be present in soil. Cyclic nitramines may be attacked at bonds so as to cause ring cleavage, which in the presence of water can lead to hydrolytic decomposition and mineralization. To prevent the decomposition reaction from undergoing sequential reduction to the corresponding nitroso derivative (nitrosation being reduction of —NO₂ to —NO), two routes may be taken: (a) attack on an —N—NO₂ bond (denitration) followed by hydration or (b) attack on a CH₂ bond (α-hydroxylation) to form unstable carbinol prior to cleavage and decomposition. The invention may be used to remediate soil and water contaminated with these explosives.

This application claims priority from U.S. Provisional Patent Application No. 60/404,147 (filed on Aug. 19, 2002) and from U.S. Provisional Patent Application No. 60/395,316 (filed Jul. 12, 2002), both of which are incorporated herein by reference.

### FIELD OF THE INVENTION

The present invention relates to degradation of nitramines, and in particular to the degradation of explosives RDX, HMX, CL20 and tetryl through chemical, biochemical, or biological attack.

### BACKGROUND OF THE INVENTION

Hexahydro-1,3,5-trinitro-1,3,5-triazine (RDX) and octahydro-1,3,5,7-tetranitro-1,3,5,7-tetrazocine (HMX) are widely used explosives recognized to severely contaminate soil and groundwater. The large scale manufacturing, use, and disposal of the cyclic nitramine explosives RDX, HMX, and CL-20), commonly used for military and commercial purposes, has resulted in severe contamination of soil and groundwater. These chemicals are toxic, mutagenic and carcinogenic to human and other biological systems.

Presently, little information is available on the initial processes involved in degradation of cyclic nitramine explosive compounds. Such information would be beneficial to elucidating the degradation pathways of these compounds, and thus to develop and optimize processes for their degradation.

Hexahydro-1,3,5-trinitro-1,3,5-triazine (RDX) is widely used for military and commercial purposes due to its superior explosive properties. The large scale manufacturing, use and disposal of RDX has resulted in elevated levels of soil and groundwater contamination. RDX and its degradation products are toxic, mutagenic and carcinogenic to humans and other biological systems. Hence there is an urgent need to remove it from the contaminated sites. Literature is available regarding the transformation of RDX under anaerobic and aerobic conditions, but little information is available regarding enzymatic degradation. Nitroreductases, largely from bacterial strains belonging to the enterobacteriacae family, may play a role in the anaerobic bacterial transformation of RDX. Until now, little has been known about the initial enzymatic processes involved in the transformafion of RDX and its products.

RDX is a highly oxidized compound. A successful initial enzymatic attack on its nitro group(s) destabilizes the inner C—N bonds and leads to ring cleavage and spontaneous decomposition, as has been demonstrated for its chemical decomposition. Methylenedinitramine has been considered a key RDX ring cleavage product in anaerobic sludge, and may have been produced by three different routes, for example initial hydrolytic cleavage of a C—N bond, hydroxylation at C-H bond and/or via formation of hexahydro-1-nitroso-3,5-dinitro-1,3,5-triazine (MNX). However, the nature of enzyme(s) involved is not clear.

It is therefore necessary to develop effective routes for degradation RDX, HMX, and other such explosives, and to optimize procedures for their degradation and mineralization, so as to optimize the bioremediation processes for the treatment of contaminated soil and groundwater.

### REFERENCES

Anusevicius et al., (1998) FEBS Lett. 436, 144-148.
Arrowsmith et al., J. Am. Chem. Soc., 1991, 113, 172.
Behrens et al., Phys. Chem. 1991, 95, 5838.
Bishop et al. Ind. Eng. Chem. Res. 1999, 38, 2254.
Bose et al. Wat. Res. 1998, 32, 1005.
Brown et al., J. Am. Chem. Soc. 1992, 114, 3092.
Chapman et al., Tetrahedron, 52 (1996) 9655-9664.
Coleman et al. Soil Biol. Biochem. 1998, 30, 1159.
Croce, et al. J. Org. Chem. 1979, 44, 2100.
Druckrey, Xenobiotica, 1973, 3, 271.
Fournier et al. J. Appl. Environ. Microbiol. 2002, 68, 166.
Haas et al. J. Anal. Chem. 1990, 338, 41-45.
Halasz et al., (2002) J. Environ. Sci. Technol. 36, 633-638
Hannink et al., (2001) Nature Biotechnol. 19, 1168-1172
Hawari et al., Chemosphere 1996, 32, 1929.
Hawari (2000) in: Biodegradation of nitroaromatic compounds and explosives (Spain et al., Eds) pp 277-310, Lewis Publishers, Boca Raton, Fla.
Hawari, Spain et al., (2000) Appl. Environ. Microbiol. 66, 2652-2657
Hawari et al., Environ. Sci. & Technol. 2001, 35, 70.
Heilmann et al., (1996) Environ. Sci. Technol. 30, 1485-1492
Hoffsommer et al., (1977) J. Phys. Chem. 81, 380-385
Huang et al., Appl. Environ. Microbiol. 66 (2000) 1474-1478.
Khan et al., J. Biol Chem. 277 (2002) 21906-21912.
Kaplan et al. Arch. Biochem. Biophys. 132 (1969) 91-98.
Kaplan, Curr. Opin. Biotechnol. 1992, 3, 253.
Kitts et al, (2000) Can. J. Microbiol. 46, 278-282
Kubose et al. Technical Report 77-20, ADA 042199, Naval Surface Weapons Center, White Oak Laboratory, Silver Spring, Md., 1977.
Lindsay et al., J. Biol. Chem 275 (2000) 36665-36670.
Lynch et al., Chem. Eng. Data. 2001, 46, 1549.
Madigan et al., Biochemical Soc. Trans 22 (1993) 57S.
March, Advanced organic chemistry. Third ed., Wiley-Interscience Publication, John Wiley & Sons, New York, US, 1985. pp 784-785.
McCormick et al., Appl. Environ. Microbiol. 1981, 42, 817.
McLellan et al., in: Health advisory for octahydro-1,3,5,7-tetranitro-1,3,5,7-tetrazocine, Technical Report PB90-273533, Office of Drinking Water, U.S. Environmental Protection Agency: Washington D.C., 1998.
Melius, In: Chemistry and Physics of Energetic Materials; Bulusu S. N. Ed.; Kluwer Academic Publishers, Netherlands, 1990; pp 21-49.
Myler et al. In Environmental Biotechnology for Waste Treatment; (Sayler et al., Eds.) Plenum Press. N.Y, 1991; pp137-146.
Nielsen et al., Tetrahedron. 1998, 54, 11793.
Oh et al., Environ. Sci. Technol. 2001, 35, 4341.
Okemgbo et al, J. Chromatogr. A. 1999, 844, 387.
Peyton et al, CERL Technical Report 99/93, (www.CECER.Army/TechReports),1999.
Regan et al., Biotechnol. Lett. 16 (1994) 1081-1086.
Ritter et al. Chem. Res. Toxicol. 11(1998) 1361-1367.
Robidoux et al. Environ. Toxic. Chem. 2000, 19, 1764.
Robidoux et al. Environ. Pollut. 2001, 111, 283.
Shah et al.,(1996) Biochem. Biophys. Res. Commun. 220, 563-568.
Sheremata et al., Environ. Sci. Technol. 2000, 34, 3384.
Summers Anal. Chem. 1990, 62, 1397.
Talmage et al., (1999) Rev. Environ. Contam. Toxicol. 161, 1-156.
Tedeschi et al., J. Biol. Chem. 270 (1995) 2512-2516.
Turro, Modem Molecular Photochemistry, Benjamin Cumming Publishing Co., 1978, ch10, pp 362.
Xia et al., Eur. J. Biochem. 268 (2001) 1486-1490.
Yinon (1990) in: Toxicity and metabolism of explosives. CRC Press, Inc., Boca Raton, Fla.
Zhao et al., Chinese Sci. Bull. 1996, 41, 574.
Zhao et al., Chem. Phys. 1988, 88, 801.
Zoh et al., Wat. Res. 2002, 36, 1331.

### SUMMARY OF THE INVENTION

It is an object of the present invention to provide processes for degradation of nitramines. These processes employ chemical, biochemical, or biological means to degrade or mineralize nitramine explosives.

According to the invention there is provided a process for degradation of cyclic nitramines comprising the steps of: attacking a first N—NO₂ group in the presence of water resulting in denitration of the first N—NO₂ group; and subsequently effecting ring cleavage. Optionally, a second N—NO₂ group may be attacked after denitration of the first N—NO₂ group, resulting in a second denitration prior to effecting ring cleavage. The nitramines may be monocyclic or polycyclic nitramines.

The invention also relates to a process to detect decomposition of hexahydro-1,3,5-trinitro-1,3,5-triazine (RDX) comprising detection of 4-nitro-2,4-diaza-butanal (4-NDAB) as a decomposition product.

The invention further relates to a process for preventing sequential reduction of cyclic nitramine explosives to nitroso-derivatives comprising the steps of: attacking a first N—NO₂ group in the presence of water resulting in denitration of the first N—NO₂ group; and subsequently effecting ring cleavage.

Further, according to the invention there is provided a process for degradation of cyclic nitramines comprising the steps of: effecting α-hydroxylation of a —CH₂ bond to form unstable carbinol; and subsequently effecting ring cleavage. The α-hydroxylation may be effected by exposure to diaphorase. Subsequently, this reaction may be followed by reduction of an —NO₂ group to —NO, and denitration prior to ring cleavage.

The invention further relates to a process for preventing sequential reduction of cyclic nitramine explosives to nitroso-derivatives comprising the steps of α-hydroxylation of a —CH₂ bond to form unstable carbinol; and subsequently effecting ring cleavage.

The invention additionally provides a process for degradation of 4-nitro-2,4-diazabutanal comprising exposure to an attack selected from the group consisting of anaerobic microbial attack, anaerobic enzymatic conditions, and chemical hydrolysis. In a preferred embodiment said attack comprises an attack selected from the group consisting of exposure to one or more of diaphorase, cytochrome P450, xanthine oxidase and nitrate reductase; exposure to a microbe selected from the group consisting of Clostridium kluyveri, Rhodococcus sp., and Aspergillus niger; and alkaline hydrolysis.

This invention has environmental applications for the optimization and control of treatment processes, whether chemical, biochemical, or biological means, developed for the degradation of explosive compounds. Advantageously, any institute, government agency, or private company that is concerned about the contamination of soil, subsurface soil, sediment and ground-water with toxic and/or mutagenic explosive compounds will benefit from this invention.

### BRIEF DESCRIPTION OF THE DRAWINGS

Preferred embodiments of the present invention will now be described, by way of example only, with reference to the attached Figures.

FIG. 1 shows representative structures of various cyclic nitramines.

FIG. 2 illustrates a LC/MS (ES−) chromatogram of RDX and MNX transformation by nitrate reductase at pH 7. Relative abundance (%) is shown. A and C show RDX and MNX, respectively, at 0 min. B and D show RDX and MNX, respectively, after 60 min, thereby showing the formation of methylene dinitramine.

FIG. 3 provides a time course of nitrate reductase catalyzed RDX transformation, ▪ RDX; □ Methylenedinitramine; ∘ N₂O; ● NADPH; ▾HCHO. Relative standard deviations are within 7%.

FIG. 4 is a schematic of a possible transformation pathway of RDX as initiated by nitrate reductase. Carbons are numbered to show the location of bond cleavage.

FIG. 5 provides Lineweaver-Burk's plots showing nitrate as the competitive inhibitor of nitrate reductase catalyzed RDX transformation. (●) 0 μM; (∘) 20 μM; (▾) 100 μM nitrate.

FIG. 6 shows NMR (DMX-600 and AV-400) and elemental analysis for C₂H₅N₃O₃.

FIG. 7 shows a proposed pathway of diaphorase catalyzed RDX transformation based on stoichiometry of metabolites recovered and NADH consumed. Primary reactions involve RDX reduction to RDX anion radical I that undergoes denitrohydrogenation to produce III. Secondary reactions involve ring cleavage and spontaneous decomposition in water. Intermediates shown inside brackets were not detected.

FIG. 8 illustrates LC/MS (ES−) chromatogram of RDX and diaphorase reaction showing methylenedinitramine as a key RDX intermediate. A, 0 hour reaction; B, 3 hour reaction.

FIG. 9 shows a time course of diaphorase catalyzed RDX transformation with simultaneous production of intermediate and end-products. (●) RDX; (∘) NADH; (▾) N₂O; (∇) HCHO; (▪) Nitrite; (□) Methylenedinitramine. Standard deviations were within 6% of the absolute mean values (n=3).

FIG. 10 shows the effect of oxygen on diaphorase catalyzed RDX transformation. A, anaerobic conditions; B, aerobic conditions. 100% RDX transformation activity was equivalent to 47 μmoles RDX transformed h⁻¹mg⁻¹ protein.

FIG. 11 illustrates the role of FMN in RDX transformation activity of diaphorase. A, native enzyme; B, apoenzyme (deflavo form); C, apoenzyme reconstituted with 200 μM FMN. 100% activity was equivalent to 41 μmoles RDX transformed h⁻¹mg⁻¹ protein.

FIG. 12 shows concentration dependent reconstitution of diaphorase by FMN. Symbols: (●) % restored activity of diaphorase; (∘) FMN (commercially available) catalyzed RDX transformation. 100% activity was equivalent to 41 μmoles RDX transformed h⁻¹mg⁻¹ protein.

FIG. 13 illustrates a proposed degradation routes of RDX by Rhodococcus sp strain DN22.

FIG. 14 provides a GC/MS (PCI) chromatogram of RDX after 2 h of photolysis at 350 nm (A) and mass spectrum of denitrated intermediate (II) at 12.7 min (B).

FIG. 15 shows an HPLC/UV chromatogram of RDX. A: before photolysis, B: after 3 h of photolysis at 350 nrn.

FIG. 16 provides an LC/MS (ES−) spectra of RDX degradation products I, IV, V, VI and VII observed during photolysis at 350 nm in aqueous solution.

FIG. 17 illustrates a time course of RDX photodegradation in aqueous solution at 350 nm. A: N—containing products. B: C—containing products. Error bars are based on triplicate measurements.

FIG. 18 illustrates possible decomposition routes of RDX following its initial denitration by photolysis at 350 nm in an aqueous solution: a comparison with Rhodococcus sp. strain DN22 (FIG. 17). Path (a): cleavage following one denitration step and path (b): cleavage following second denitration step.

FIG. 19 provides a schematic representation of the alkaline hydrolysis of RDX (Refs. 16-18).

FIG. 20 provides a time course of the alkaline hydrolysis of RDX at pH 10. Data points are the mean and error bars the standard deviation (n=3).

FIG. 21 provides typical LC/MS chromatograms of intermediates formed during hydrolysis of cyclic nitramines RDX, MNX and HMX in aqueous media at pH 10. (A) RDX after 8 days (B) MNX after 1 day (C) HMX after 15 days. Note that 4-nitro-2,4-diaza-butanal (4-NDAB) appears as a key product in each case.

FIG. 22 provides a time course of the alkaline hydrolysis of MNX at pH 10. Data points are the mean and error bars the standard deviation (n=3).

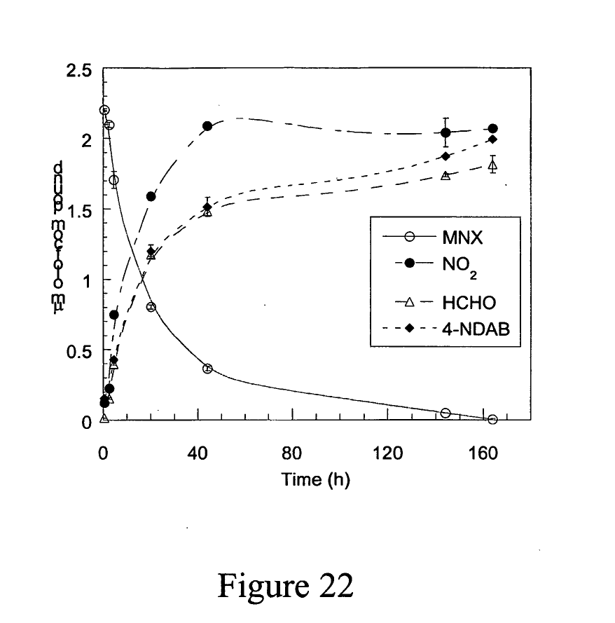

FIG. 23 illustrates LC/MS chromatograms of RDX and the early intermediates formed during its degradation in acetonitrile: water (70:30% v/v) at pH 12.3. (A): In the absence of hydroxypropyl-β-cyclodextrin (HPPCD) and (B): in the presence of HPβCD (3% w/v). In the case of (B), the chromatogram was obtained using a SynergiPolar™-RP column (4.6 mm ID by 15 cm; Phenomenex, Torrance, Calif.) at 25° C., and eluted using a methanol:water gradient. III: 4,6-dinitro-2,4,6-triaza-hexanal, IV: 5-hydroxy-4-nitro-2,4-diaza-pentanal.

FIG. 24 illustrates a LC-MS (ES) mass spectra of 4,6-dinitro-4,6-diaza-hexanal (III) (A), 5-hydroxy-4-nitro-2,4-diaza-pentanal (IV) (B) and its ¹⁵N labeled analog ¹⁵N-IV (C).

FIG. 25 shows a major route for the alkaline hydrolysis of RDX. Bracketed compounds were not observed.

FIG. 26 shows a minor route in the alkaline hydrolysis of RDX reaction intermediate III. Bracketed compounds were not observed.

### DETAILED DESCRIPTION OF THE INVENTION

The invention provides chemical, biochemical, and biological processes for RDX, HMX and CL-20 and tetryl transformation, leading to complete mineralization of these explosives. The invention involves either chemical processes which may be chemical, for example photolysis or alkaline hydrolysis, biochemical, for example using specific enzymes such as diaphorase, cytochrome P450, xanthine oxidase, nitrate reductase, and nitrate oxidoreductase or may be biological, for example using specific microbes intermixed with a sample containing the nitramines explosives. For example, Clostridium kluyveri, Rhodococcus sp. (various strains, including DN22, A-BRI, and 11Y), and Aspergillus niger may be used.

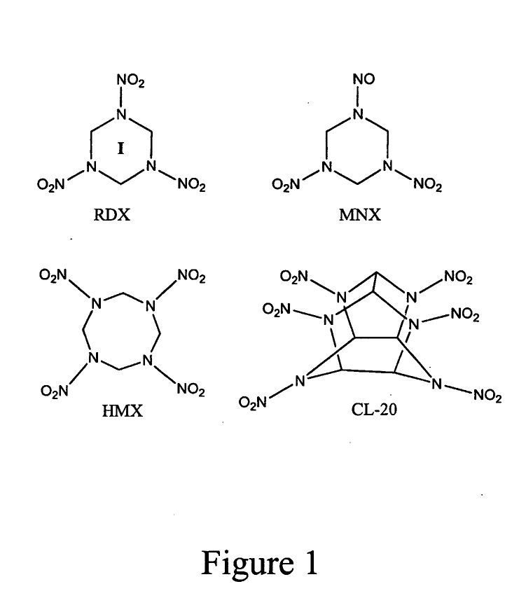

FIG. 1 shows the structures of various cyclic nitramines which may be degraded according to the invention, including RDX, MNX, HMX and CL-20. Advantageously, the invention allows degradation of these and other cyclic nitramines into inert or non-explosive products.

According to the invention, processes are used on cyclic nitramine explosives (e.g., RDX, HMX, and CL-20) to stop the explosives from undergoing sequential reduction to the corresponding di-nitroso, tri-nitroso, and in the case of HMX, tetra-nitroso derivatives. An initial denitration step (enzymatic, microbial or chemical) on RDX and HMX decreases or eliminates sequential reduction of the nitro groups to the corresponding nitroso derivatives in this family of cyclic nitramine explosives. This step leads to spontaneous decomposition in water or wet soil. The conditions required to selectively facilitate the desired decomposition reactions can be created by varying parameters including: types of enzyme used, the physiological conditions of the microbes employed, the chemical reagents utilized, photodegradation conditions, and the redox potential of the system.

The invention allows for custom design of remediation strategies revolving around the selected method of initial attack to yield a rapid, efficient, and spontaneous decomposition of RDX and HMX in water or in wet soils. The process of RDX and HMX transformation according to the invention shows that the intermediate metabolites and end products of degradation will vary depending upon the nature of initial attack on these molecules.

The invention further relates to a system for preventing the sequential reduction of these explosives to their nitroso-derivatives, but instead selectively promotes a mechanism of initial denitration attack that rapidly, efficiently and spontaneously produces a well defined set of harmless end products. Advantageously, this results in safe conversion of these highly toxic cyclic nitramines to gaseous products.

The invention allows for degradation of 4-nitro-2,4-diazabutanal by exposure to an attack such as anaerobic microbial or enzymatic attack, or by chemical hydrolysis. This could be accomplished, for example, by moving a reacted mixture of anaerobic sludge to conditions used to degrade RDX in sludge. Alternatively, the reacted mixture can be hydrolysed under relatively high pH conditions.

Processes are described herein for selective degradation of nitramine explosives. The invention is based on the discovery that nitramine explosives, for example, RDX, HMX, CL20 and tetryl can be both enzymatically and chemically attacked at several bonds. The outcome of the attack may depend on the initial point (bond) of attack on these molecules. This observation was used in this invention to develop chemical, biochemical, and biological reaction on these compounds to obtain effective mineralization to nitrous oxide and carbon dioxide. The invention has important implications in the field of remediation of soil and water contaminated with these explosives.

Cyclic nitramines have distinctive bonds (functional groups) for possible chemical and enzymatic attack. These bonds include —NO₂, —N—NO₂, —CH₂—N and -CH₂. According to the invention, among the possible sites of attack, two particular reactions are responsible for the rapid and effective ring cleavage, which in the presence of water can lead to effective hydrolytic decomposition and mineralization. The invention allows control of such reactions, to prevent functional groups from undergoing sequential nitrosation (reduction of —NO₂ to —NO), but to instead enhance attack on either the —N—NO₂ (denitration followed by hydration) or the CH₂ (α-hydroxylation) to form and unstable carbinol prior to cleavage and decomposition. These latter reactions are important for effective mineralization of nitramine based explosives. The invention allows the initial reaction to be conducted under both anaerobic and aerobic conditions in soil and in water biochemically (enzymatically), biologically (microbially) and chemically (photochemically or with alkaline hydrolysis). As far as low water solubility is concerned, cosolvents may be added to the reaction conditions to enhance solubitity and to catalyze degradation. Such co-solvents include additives such as cyclodextrins to enhance solubility and also to catalyze degradation.

As used herein, the term “chemical” means any chemical reaction, whether caused by photolytic, hydrolytic, inorganic chemical catalysis or other chemical reactions. The term “biochemical” as used herein refers to a reaction caused by any biochemical mechanism, for example thorough the use of an enzyme, enzyme equivalent, or an organic catalyst. The term “biological” as used herein refers to a reaction caused by the use of a biological entity, such as by a microbe or other lower life form, or a portion thereof.

The invention is a model system for understanding the mechanism of initial enzymatic attack on cyclic nitramines such as RDX, and its subsequent degradation by an enzyme.

Nitrate reductases are ubiquitous enzymes in diverse groups of microorganisms, especially denitrifying bacteria, and their physiological role is to reduce nitrate to nitrite via a two electron transfer. Hence there is a great possibility that a nitrate reductase, a nitroreductase, or a similar enzyme may be one of the key enzymes responsible for RDX transformation in anaerobic sludge. No information existed until now regarding nitrate reductase reaction with cyclic nitramine compounds such as RDX.

In this invention, a variety of enzymes may be used. For example, a nitrate reductase (EC 1.6.6.2) from a fungus Aspergillus niger may be used to transform RDX under anaerobic conditions. Other similar nitrate reductases or nitroreductases from other microorganisms would also work.

The invention involves the detailed enzymatic mechanisms for transformation of RDX, HMX and CL-20, leading to their substantially complete mineralization, based on results obtained through experiments involving microbial enzymes, such as nitrate reductase. Reactions with cyclic nitramine explosives (e.g., RDX, Mx, and CL-20) may be carried out to prevent the compounds from undergoing sequential reduction to the corresponding di-, tri-, and in the case of HMX, tetra-nitroso derivatives. These derivatives are extremely toxic and persist in the environment for longer periods.

Biotransformation of one nitro group is sufficient for ring cleavage of cyclic nitramine compounds and ensues in subsequent decomposition. Experimental conditions required to selectively facilitate the desired decomposition reactions may be created by varying parameters including: the type(s) of enzyme used, the physiological conditions of the microbes employed and the redox potential of the systems.

The present invention serves to safely convert these highly toxic cyclic nitramines to harmless products and is useful for designing remediation strategies revolving around the selected method of initial attack to yield a rapid, efficient, and spontaneous decomposition of RDX and HMX in water or in wet soils.

The detailed mechanisms of RDX and HMX transformation show that the intermediate metabolites and end products of the degradation processes vary depending upon the nature of initial attack on these molecules. The invention encompasses a system to prevent the sequential reduction of cyclic nitramines to their highly toxic, recalcitrant, multi-nitroso derivatives. Instead, the route of initial attack according to the invention produces harmless end products with relative rapidity, efficiency and spontaneity.

The processes according to the invention may be used to degrade cyclic nitramine explosives present in a natural environment, such as in water or soil. In order to conduct such remediation activities, soil samples or water samples can be removed from the site for treatment, or they may be treated on site. In the case of treating soil, an area may be flooded, to form a soil slurry or a flooded biopile. Further, for long-term remediation projects, plants may be introduced into a contaminated area in which microbial species have been added to root nodules, or in which modified bacteria (such as Rhizobium species) containing appropriate enzymes to conduct the inventive processes has been added. In this way, phytoremediation of a region may be effected by enzymatic conversion of cyclic nitramines in the soil by the root tips of such plants. All such remediation strategies fall within the scope of the invention.

### EXAMPLE 1

### Biotransformation with Nitrate Reductase from Aspergillus niger

In this Example, biotransformation of RDX with nitrate reductase derived from Aspergillus niger is exemplified.

Chemicals. Commercial grade RDX (purity >99%) was provided by Defence Research Establishment, Valcartier, Quebec, Canada MNX of either 95% or 99% purity was used. For the experiment describing MNX biotransformation with nitrate reductase, MNX (>99% purity) was obtained from SRI International (Menlo Park, Calif.). Aspergillus niger NAD(P)H: Nitrate oxidoreductase (EC 1.6.6.2), NADPH and formaldehyde were obtained from Sigma Chemicals, Canada. Methylenedinitramine was obtained from the rare chemical department of Aldrich, Oakville, ON, Canada. Hydrazine and formamide were purchased from Aldrich, Canada. Standard nitrous oxide (N₂O, 980 ppm by mole) was obtained from Scott Specialty Gases, Sarnia, ON, Canada.

Enzyme preparation and assays. Lyophilized enzyme is suspended in potassium phosphate buffer (50 mM) at pH 7.0 and washed thrice with 2.5 ml of buffer using Biomax-5K membrane centrifuge filter units (Sigma chemicals). The washed enzyme is then suspended in 0.5 ml of buffer and the protein concentration is measured by bicinchoninic acid (BCA) kit (Sigma chemicals) using bovine serum albumin as standard. The native enzyme activity is estimated spectrophotometrically at 340 nm as the rate of oxidation of NADPH in the presence of nitrate.

Nitrate reductase catalyzed RDX transformation is performed under anaerobic conditions in 6 ml glass vials containing one ml of reaction mixture sealed under an atmosphere of argon. The above assay mixture contained RDX (or MNX) (100 μM), NADPH (300 μM) and one mg enzyme (equivalent to 0.25 native units) in potassium phosphate buffer (50 mM) at pH 7.0 and 30° C. Three different controls are prepared as follows: 1) RDX and NADPH in buffer (i.e. without enzyme); 2) RDX and enzyme in buffer (i.e. in the absence of NADPH); and, 3) RDX only in buffer. The samples from liquid and gas phases are withdrawn periodically to analyze for RDX and transformation products as described below. The enzymatic transformation rate of RDX is expressed as μmoles RDX transformed h⁻¹mg⁻¹protein.

To study the fate of formamide and hydrazine in the reaction mixture, each chemical (50 μM) is incubated separately with nitrate reductase and NADPH for one hour at pH 7.0 and 30° C. under the same conditions as described above. The residual compounds and their products are analyzed as stated below.

Analytical procedures. RDX is analyzed by a reversed phase-high pressure liquid chromatograph (RP-HPLC) connected to photodiode array (PDA) detector (λ_{254 nm}). Samples (50 μl) are injected into a Supelcosil LC-CN column (4.6 mm ID×25 cm) (Supelco, Bellafonte, Pa.) and the analytes are eluted using methanol/water gradient at a flow rate of 1.5 ml/min. The MNX and methylenedinitramine are analyzed by a Micromass bench-top single quadrupole mass detector attached to a Hewlett Packard 1100 series HPLC system equipped with a photodiode array detector. Ionization is carried out in a negative electrospray ionization mode ES(−). Their identities are confirmed by comparison with a reference standard.

Hydrazine is analyzed by a HPLC system equipped with a Waters model 600 pump (Waters Associates, Milford, MA), a 717 plus autosampler, a Hamilton RPX-X200 analytical cation exchange column (250 mm×4.1 mm), a Waters post column reaction module with a Waters reagent manager pump, a Waters model 464 electrochemical detector with a gold-working electrode and a base resistant Ag/AgCl reference electrode.

The eluent is 6% v/v acetonitrile in 0.005 M KH₂PO₄ solution in deionized water. The eluent is degassed by continuous helium sparging before and during use. The post-column reaction solution is 0.1 M NaOH solution in deionized water. The operating parameters for the system are: eluent flow rate, 1.0 ml/min.; temperature, 30° C.; injection volume, 25 μl; flow-rate of post-column reaction solution, 250 μl/min.; working electrode cleaning potential, 500 mV (0.333 sec); pretreatment potential, −350 mV (0.333 sec) and measuring potential, 100 mV in DC mode.

Formamide is analyzed by a Micromass bench-top single quadrupole mass detector attached to an HPLC system equipped with a photodiode array detector and a synergi polar-RP column (4.6 mm ID×15 cm) (Phenomenex, Torrance, Calif.) at 25° C. The solvent system is a methanol/water gradient (10-90% v/v) at a flow rate of 0.75 ml/min. For mass analysis, the ionization is carried out in a positive electrospray ionization mode ES⁺ producing mainly the [M+H] mass ions. The electrospray probe tip potential is set at 3.5 kV with a cone voltage of 35 V at an ion source temperature of 150° C.

A. niger nitrate reductase catalyzes the transformation of RDX in the presence of NADPH as electron donor. One embodiment for the transformation is under anaerobic conditions at pH 7.0 and 30° C. (data not shown). The disappearance of RDX is accompanied by the formation of MNX and the ring cleavage product methylenedinitramine (FIG. 1, parts A and B).

In control experiments, no degradation is observed except in control number 1 which contained NADPH and RDX (see experimental section) where a negligible RDX loss (<3% of total RDX degradation) is observed in one hour of reaction time.

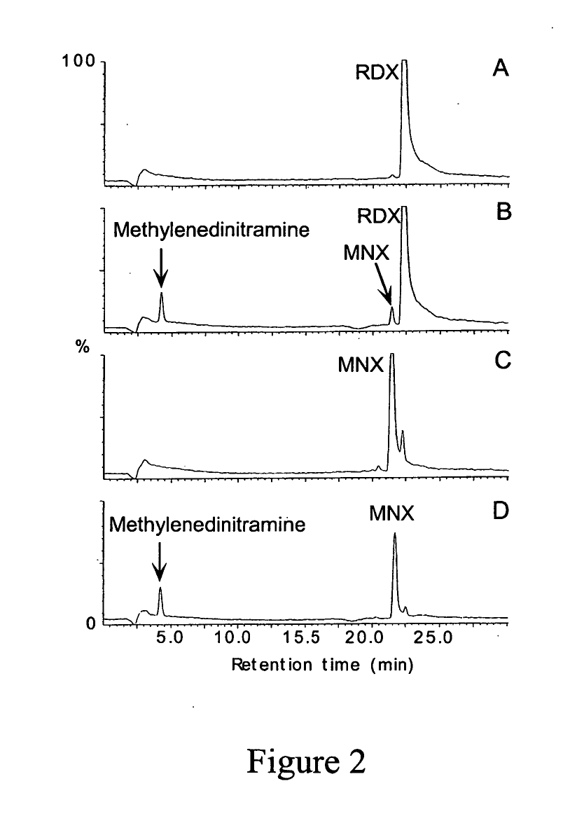

To further prove that RDX is first reduced to MNX and the latter undergoes further reduction to finally produce methylene dinitrarnine, (O₂NNH)₂CH₂, the standard MNX is incubated with nitrate reductase under similar reaction conditions as those of RDX biotransformation (see above). As a result, we observed the formation of MNX forms (O₂NNH)₂CH₂MNX (FIG. 2, parts C and D).

FIG. 2 is a typical LC/MS (ES−) chromatogram obtained after incubating RDX and MNX in separate vials with nitrate reductase for 60 minutes under the optimal reaction conditions. Methylenedinitramine and MNX are identified by comparing their chromatographic retention times and LC/MS (ES−) spectra with those of reference standards. MNX exhibits a retention time of 20.25 min and produces characteristic mass fragments at 46 Da and 251 Da, representing NO₂ and an unidentified adduct (205 Da from [M−H] of MNX and 46 Da), respectively.

Methylenedinitramine exhibits a characteristic mass data at 61 and 135 Da, representing NHNO₂ group and the [M−H] mass ion of the parent compound.

Interestingly, in FIG. 2 there is the absence of the dinitroso (DNX) and trinitroso (TNX) products of RDX. None of the products in FIG. 2 persist and their disappearance is accompanied by the accumulation of formaldehyde (HCHO), ammonium ion (NH₄⁺) and nitrous oxide (N₂O).

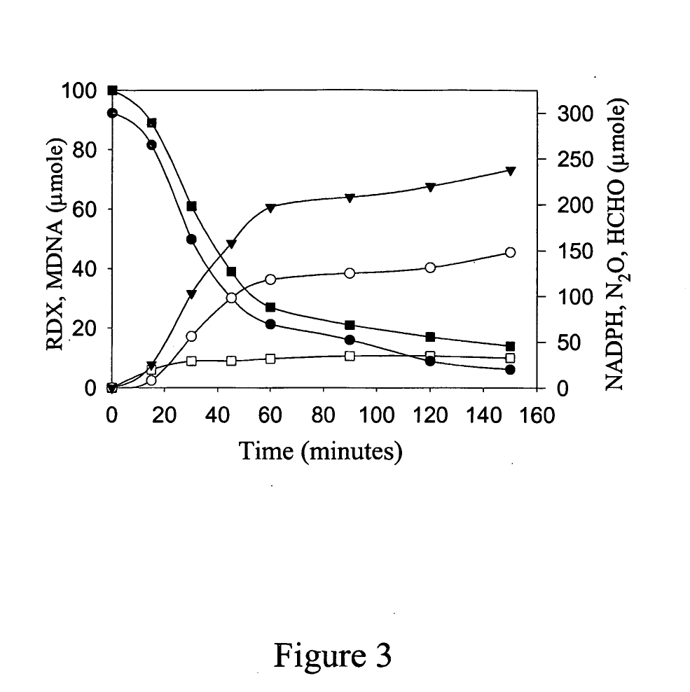

Stoichiometry and Pathway. In the time course study, methylenedinitramine is observed as the prime intermediate and N₂O and HCHO are the major end products (FIG. 3). Throughout the reaction course the concentration of methylenedinitramine is low and does not exceed 10 μmoles because it is quite unstable at pH 7.0 and is rapidly decomposed into N₂O and HCHO. To calculate the stoichiometry of RDX biotransformation with the enzyme, the phenomenon of quantitative decomposition of methylenedinitramine into two molecules of N₂O and one molecule of HCHO is used.

Table 1 illustrates carbon and nitrogen mass balance and stoichiometry of metabolites produced during RDX transformation catalyzed by nitrate reductase from Aspergillus niger after 150 minutes of reaction. Table 1 shows that 86 μmoles of RDX produces 148 μmoles N₂O and 10 μmoles methylenedinitramine (equivalent to 20 μmoles N₂O). This demonstrates that for each reacted molecule of RDX only one molecule of methylenedinitramine, containing two N—NO₂ groups, is produced.

TABLE 1
Metabolites Produced During RDX Transformation Catalyzed by Nitrate
Reductase from Aspergillus niger

| | | Concentration | | |
|---|---|---|---|---|
| | | of Reactants/- | | |
| Reactants/ | | metabolites | % Carbon | % Nitrogen |
| Metabolites | | (μmole) | recovery* | recovery* |
| 1. | RDX | 86 | 100 | 100 |
| 2. | NADPH | 280 | NA | NA |
| 3. | Methylenedinitramine | 10 | 3.87 | 7.75 |
| | (MDNA) | | | |
| 4. | Formaldehyde | 238 | 92.27 | NA |
| | (HCHO) | | | |
| 5. | Nitrous oxide | 148 | NA | 57.37 |
| | (N₂O) | | | |
| 6. | Ammonium ion | 88 | NA | 16.98 |
| | +UZ,27/31 (NH₄⁺) | | | |

| | | |
|---|---|---|
| Total % mass recovery | 96.14 | 82.10 |

*Calculated from the total carbon and nitrogen mass in the transformed RDX (86 μmole);
NA, not applicable;
Standard deviations are within 7% of the mean absolute value (n = 3).

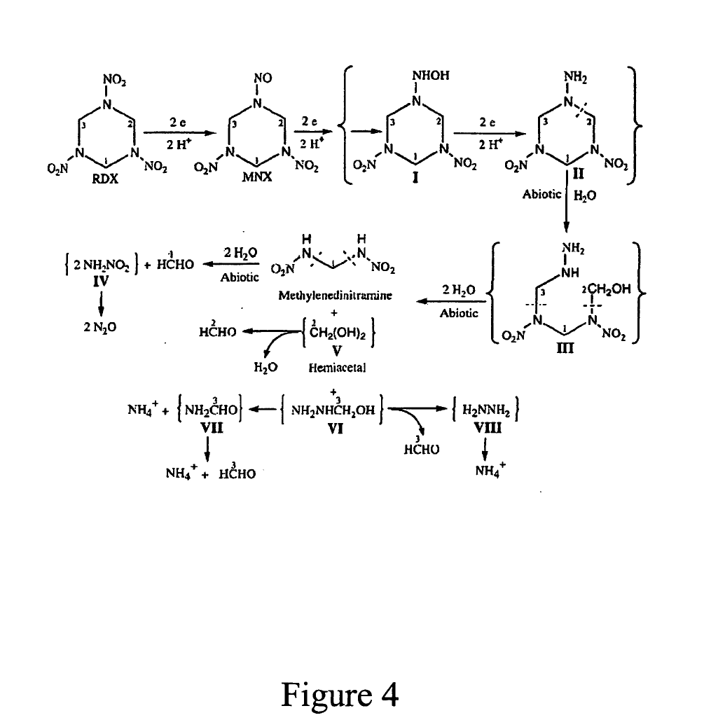

A part of the remaining N—NO₂ group in RDX leads to the formation of ammonium ion (88 μmoles). The total nitrogen mass balance is 82.10% and is distributed between N₂O (57.37%), methylenedinitramine (7.75%), and ammonium ion (16.98%) (Table 1). The carbon mass balance is 96.13% and is distributed between HCHO (92.27%) and methylenedinitramine (3.87%) which indicates that all the recovered carbon is in the form of HCHO. Hence, one RDX molecule produces three HCHO molecules (FIG. 4).

It is difficult to quantify MNX since it exists only in trace amount throughout the RDX reaction time course. It is believed that only MNX (with two N—NO₂) produces methylenedinitramine (with two N—NO₂) and that neither DNX (with only one N—NO₂) nor TNX (with no N—NO₂) can act as a precursor to methylenedinitramine. In addition, as aforementioned, neither of the two compounds is detected. Finally, for each reacted RDX molecule, roughly three molecules of NADPH, representing 6 electrons, are consumed (Table 1).

Based on this stoichiometry, we propose a degradation pathway for RDX as shown in FIG. 4. Roman numerals used in reference to this Example indicate intermediates having numerals within FIG. 4. RDX is believed to undergo reduction in three sequential steps to first produce MNX, followed by the formation of the hydroxylamine derivative RDX-NHOH (I), which then reduces further to the amine metabolite RDX-NH₂(II). Neither of the two hypothetical compounds is detected. If formed, both I and II are expected to be unstable in water and might undergo rapid ring cleavage to produce an unstable hydroxyalkylnitramine product (III) (FIG. 4) which is unstable in water and thus III will undergo rapid decomposition to produce methylenedinitramine, HCHO and another hypothetical compound possibly methanolhydrazine (VI) (NH₂NHCH₂OH).

The latter, being a form of a-hydroxylamine should also decompose in water since it is not possible to detect it. The decomposition of VI is presumed to proceed by two possible routes to produce HCHO and NH₄⁺ i.e. via formation of formamide and/or hydrazine (FIG. 4). However, these two compounds do not accumulate and are therefore undetectable. For example, when either formamide (50 μM) or hydrazine (50 μM) is incubated with nitrate reductase under similar reaction conditions to those described for RDX, roughly 50% of the formamide is converted to HCHO and NH₄⁺, and 40% of the hydrazine to NH₄+.

82.10% of the total nitrogen mass content of RDX is accounted for, and it is believed that the missing 18% nitrogen mass exists in the form of an unidentified compound that might react with other components, as aforementioned. In particular, hydrazine is a very reactive compound and it is known to polymerize with HCHO. DNX and TNX are excluded from the RDX biotransformation pathway (FIG. 4) based on the observation that neither is detected by LC/MS (ES−). Furthermore, neither of the two nitroso products can produce methylenedinitramine.

In contrast, the stoichiometric formation of methylenedinitramine (one μmole of RDX produces one μmole of methylenedinitramine) confirms that ring cleavage proceeds via MNX (FIG. 4). In support of these findings, when MNX is incubated with nitrate reductase under the same conditions as used for RDX, the formation of methylene dinitramine (FIG. 2, parts C and D) is observed as a key ring cleavage product which also decomposes to HCHO and N₂O. Once again neither DNX nor TNX is observed.

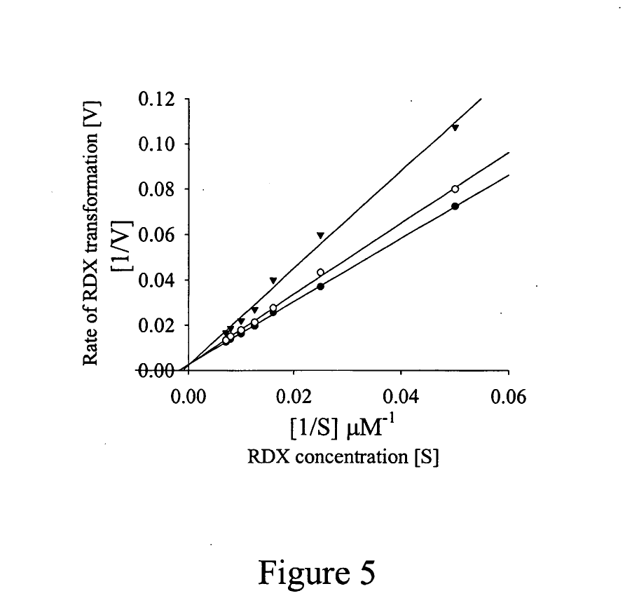

Role of Nitrate Reductase in the Initial Transformation of RDX. Eukaryotic nitrate reductases (EC 1.6.6.1-3) contain three redox cofactors i.e. FAD, heme-Fe and molybdopterin (Mo-MPT). FAD is the oxidation site for NAD(P)H and the subsequent electron transfer proceeds through a heme-Fe center to the molybdopterin which is a reduction site for nitrate. During nitrate reduction, the molybdenum (Mo) present in the Mo-MPT cofactor frequently changes its oxidation state from oxidized (Mo^{VI}) to reduced (Mo^{IV}) and vice-versa by receiving two electrons from the heme-Fe center and transferring them to nitrate. Part of the invention resides in showing that nitrate is a competitive inhibitor of RDX biotransformation, as evidenced by the Lineweaver-Burk's plots in the presence of increasing concentration of nitrate in the reaction mixture (FIG. 5). This indicates that nitrate and RDX likely share a common binding site on the enzyme (i.e. Mo-MPT cofactor).

The transformation of 86 μmoles of RDX is accompanied by the consumption of 280 μmoles of NADPH. This indicates that roughly three NADPH molecules are consumed during transformation of one RDX molecule under anaerobic conditions (Table 1). Therefore, a total of six electrons (or 3 NADPH) participate in the complete transformation of RDX. Since RDX requires an obligatory two electrons to be transformed to MNX, it is concluded that nitrate reductase catalyzes two-electron transfer to the RDX in the first reduction step to produce MNX. Thereafter, the remaining two pairs of electrons are presumably involved in the sequential reduction of MNX to RDX-NHOH (I) (1 NADPH) and RDX-NH₂ (II) (1 NADPH) (FIG. 4).

However, it is also possible that RDX-NHOH, being unstable, undergoes ring cleavage, using the last pair of electrons (1 NADPH) after the cleavage.

The transformation of cyclic nitramine compound(s) with nitrate reductase(s) has not yet been characterized elsewhere in the literature.

The results clearly demonstrate that MNX is the first reduced product of RDX formed by a two-electron reduction catalyzed by nitrate reductase. MNX undergoes further reduction followed by the ring cleavage and subsequent decomposition to produce HCHO, NH4+, and N₂O.

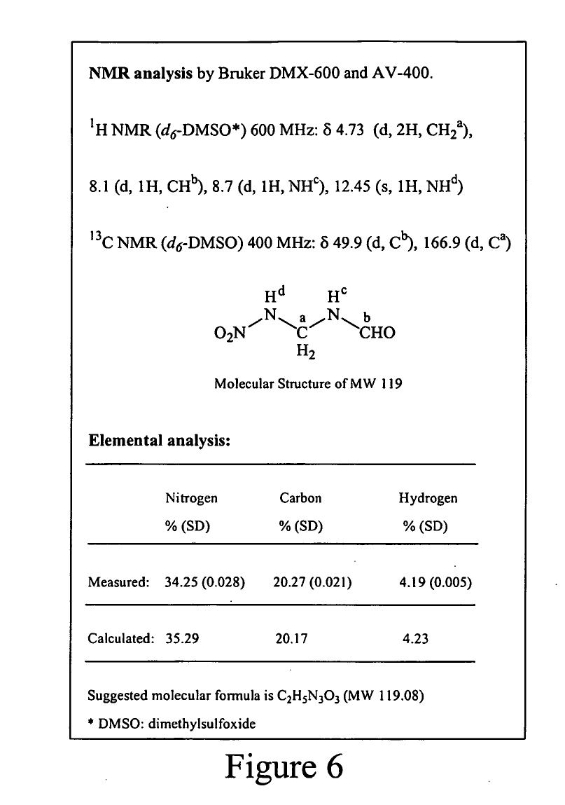

FIG. 6 shows NMR (DMX-600 and AV-400) and elemental analysis for C₂H₅N₃O₃.

### EXAMPLE 2

### RDX Biotransformation with Diaphorase from Clostridium kluyveri

Hexahydro-1,3,5-trinitro-1,3,5-triazine (RDX) can be biotransformed by anaerobic sludge via three different routes, 1: direct ring cleavage via a-hydroxylation of a —CH₂ group, 2: reduction of one of the —NO₂ groups to —NO, and 3: N-denitration prior to ring cleavage. The present Example describes biotransformation of RDX via route 3 by a diaphorase (EC 1.8.1.4) from Clostridium kluyveri using NADH as electron donor. The removal of RDX was accompanied by the formation and accumulation of nitrite ion (NO₂—), formaldehyde (HCHO), ammonium (NH₄+) and nitrous oxide (N₂O). None of the RDX-nitroso products were detected. The ring cleavage product methylenedinitramine was detected as the transient intermediate. Product stoichiometry showed that each reacted RDX molecule produced one nitrite ion and the product distribution gave a carbon (C) and nitrogen (N) mass balance of 91% and 92%, respectively, supporting the occurrence of a mono-denitration step prior to the ring cleavage and decomposition. Severe oxygen mediated inhibition (92% inhibition) of RDX biotransformation and superoxide dismutase-sensitive cytochrome c reduction indicated the potential involvement of an anion radical RDX●⁻ prior to denitration. A comparative study between native- and apo- enzymes showed the possible involvement of flavin mononucleotide (FMN) in catalyzing the transfer of a redox equivalent (e/H+) from NADH to RDX in order to produce RDX●⁻ responsible for secondary decomposition.

Introduction. It has been recently reported that RDX can be easily degraded with anaerobic sludge to produce HCHO, CO₂, N₂O and NH₃ (Hawari et al., 2000), but there was no elaboration on the enzymes responsible for initiating RDX degradation. Kitts et al. (2000) have reported the reduction of RDX by a type 1 (2 electron transfer process) oxygen-insensitive nitroreductase without providing details on either products or degradation pathways. Whereas Shah and Spain et al. (1996) reported N-denitrohydrogenation of tetryl by a ferredoxin-NADP oxidoreductase (EC 1.18.1.2) using NADPH as electron donor. Example 1 shows that RDX can be transformed by a nitrate reductase from Aspergillus niger to produce MNX via a 2 e- transfer process prior to its decomposition to HCHO and N₂O.

In this Example, a flavoenzyme, diaphorase, was selected from an anaerobic bacterium Clostridium kluyveri to biotransform RDX. Clostridium species are common inhabitants of anaerobic environments and thus it is expected that diaphorase is one of the key enzymes responsible for RDX degradation by the anaerobic sludge. Previously, Clostridium species have been known to biotransform RDX and nitroaromatic explosive, 2,4,6-trinitrotoluene (TNT) (Regan et al., 1994). A carbon monoxide dehydrogenase from Clostridium thermoacticum reportedly biotransformed TNT (Huang et al., 2000). However, no detailed study has been reported so far in regard to biotransformation of RDX by a purified enzyme from Clostridium species. The diaphorase (EC 1.8.1.4) from Clostridium kluyveri is a 24 kDa flavoenzyme containing 1 mole of FMN per mole of enzyme, and it catalyzes the pyridine nucleotide dependent reduction of dyes. This enzyme catalyzed an oxygen-sensitive 1 e-reduction of nitrofluorenes (Ritter et al., 1998). The present Example illustrates the metabolites and the mechanism of RDX biotransformation catalyzed by the diaphorase (EC 1.8.1.4) from Clostridium kluyveri. This process is useful for achieving optimal mineralization of this energetic chemical under field conditions.

Chemicals. Commercial grade RDX (purity>99%) was provided by Defense Research and Development, Quebec, Canada. NADH, flavin monononucleotide (FMN). 2,6-dichlorophenol-indophenol (DPIP), superoxide dismutase (SOD, EC 1.15.1.1, from bovine erythrocytes), cytochrome c (from horse heart, MW 12,384 Da) and formaldehyde were purchased from Sigma Chemicals, Canada. Methylenedinitramine was obtained from the rare chemical department of Aldrich, Canada. Standard nitrous oxide (N₂O, 980 ppm by mole) was obtained from Scott specialty gases, Sarnia, ON, Canada. All other chemicals were of highest purity grade available.

Enzyme preparation. Diaphorase (BC 1.8.1.4) from Clostridium kluyveri was obtained from Sigma Chemicals, Canada, as a lyophilized powder. The enzyme was suspended in 50 mM potassium phosphate buffer (pH 7.0) and filtered through a Biomax-SK membrane (Sigma Chemicals) before resuspension in the same buffer. The protein concentration was measured by bicinchoninic acid (BCA) kit (Sigma Chemicals) using bovine serum albumin as standard. The native enzyme activity was estimated (as per company guidelines) spectrophotometrically at 340 nn as the rate of oxidation of NADH using 2,6-dichlorophenol-indophenol as the electron acceptor.

Biotransformation Assays. RDX biotransformation assays with diaphorase were performed in 6 ml glass vials under anaerobic conditions (with an atmosphere of argon) at pH 7.0 and 27° C. Each vial contained RDX (100 μM), NADH (150 μM) and 50 μl of the enzyme (0.5 native units) in a final volume of one ml potassium phosphate buffer (50 mM, pH 7.0). Three different controls were prepared. The first control contained RDX, NADH and buffer without enzyme; the second contained RDX, enzyme and buffer without NADH, and the third contained only RDX and buffer. The reaction time was one hour unless stated otherwise. Samples from the liquid and gas phases in the vials were withdrawn periodically to analyze for RDX and the transformed products as described below. RDX transformation activity of enzyme was expressed as μmoles of RDX transformed h⁻¹mg⁻¹ protein.

Inhibition with Oxygen. The effect of oxygen on RDX transformation activity of diaphorase was studied by performing the assays under aerobic and anaerobic conditions at pH 7.0 and 27° C. In another experiment, we determined the role of O₂ transformation to superoxide radical anion (O₂●⁻) in the inhibition of RDX biotransformation. This was done by incubating the RDX with diaphorase in the presence of NADH, cytochrome c (100 μM and superoxide dismutase (SOD) (150 μg/ml) as described previously (Anusevicius et al., 1998). The absorption of reduced cytochrome c was monitored at 550 nm.

Preparation ofapoenzyme (Deflavo Enzyme) and its Reconstitution. The deflavo form of diaphorase was prepared as described before (Madigan et al., 1993 and Tedeschi et al., 1995) with some modifications. The holoenzyme was dialyzed for 48 hours at 4° C. against a dialysis solution composed of 100 mM potassium phosphate buffer (pH 7.0), EDTA (0.1 mM), glycerol (10% v/v) and KBr (3 M). The dialysis buffer was changed every 6 hours. The reconstitution of apoenzyme was carried out in ice cold potassium phosphate buffer (pH 7.0) in the presence of glycerol (10% v/v). FMN was added at variable concentrations (0-250 μM) to the apoenzyme preparation. The unbound FMN was removed by washing the enzyme with the same buffer using Biomax-5K membrane centrifuge filter units. The enzyme activity was assayed after each addition of FMN to the apoenzyme in order to determine the concentration-dependent reconstitution of apoenzyme by the FMN.

Analytical Procedures. RDX was analyzed by a reversed phase-high performance liquid chromatograph (RP-HPLC) connected to photodiode array (PDA) detector (λ254 nm). Samples (50 μl) were injected into a Supelcosil LC-CN column (4.6 mm ID×25 cm) (Supelco, Bellafonte, Pa.) and the analytes were eluted using methanol/water gradient at a flow rate of 1.5 ml/min (Hawari et al., 2000). Methylenedinitramine was analyzed by a Micromass™ bench-top single quadrupole mass detector attached to a Hewlett Packard 1100 series HPLC system equipped with a photodiode array detector. Ionization was carried out in a negative electrospray ionization mode ES(−) producing mainly the deprotonated molecular mass ions [M−H]. Methylenedinitramine was detected as its deprotonated molecular mass ion [M−H] at 135 Da and confirmed by comparison with a reference standard (Halasz et al., 2002). Nitroso-derivatives of RDX (i.e. hexahydro-1-nitroso-3,5-dinitro-1,3,5,-triazine, MNX; hexahydro-1,3-dinitroso-5-nitro- 1,3,5-triazine, DNX and hexahydro-1,3,5-trinitroso-1,3,5-triazine, TNX), nitrite (NO2), ammonium (NH₄+), formaldehyde (HCHO) and nitrous oxide (N₂O) were analyzed as reported (Hawari et al., 2000 and Halasz et al., 2002).

Results and Discussion

RDX Biotransformation and Product Identification. Clostridium kluyveri diaphorase was found to transform RDX at pH 7.0 and 27° C. under anaerobic conditions using NADH as the electron donor.

RDX transformation with diaphorase showed the production bf methylenedinitramine as a key ring cleavage metabolite at a retention time of 4.2 minutes. None of the nitroso-RDX intermediates, particularly hexahydro-1-nitroso-3,5-diflitro-1,3,5-triazine (MNX), were detected, although such initial RDX reduced products are frequently observed during RDX degradation by anaerobic sludge. No biotransformation of RDX was observed in the control experiments that did not contain enzyme and/or NADH. The disappearance of RDX and NADH were accompanied by the formation and accumulation of HCHO, N₂O and NO₂—.

Inhibition by Oxygen. It was found that the presence of oxygen (O₂) inhibited (92%) the transformation of RDX, thereby supporting the involvement of an oxygen-sensitive step during the initial enzymatic attack on RDX. A negligible oxidation of NADH under aerobic conditions was observed within the assay time of one hour, indicating that O₂ inhibition of RDX biotransformation was not due to the depletion of NADH. It was observed that diaphorase did not catalyze the electron transfer from NADH to O₂ and this observation was similar to the one reported by Kaplan et al. (1969) using the same enzyme. Based on these observations, inhibition of RDX transformation by O₂ is most probably due to quenching of an unpaired electron from the RDX anion radical (RDX●⁻) by O₂. Such a reaction would revert RDX●⁻, an initial transformation product, to its original structure RDX producing, instead, O₂ and thus preventing the molecule from ftirther decomposition as was the case with tetryl (Shah et al., 1996 and Anusevicius et al., 1998). RDX●⁻ appears to be formed via an initial electron transfer process catalyzed by diaphorase. Ritter et al. (1998) also reported the reduction of a nitro group in nitrofluorene compounds via an O₂-sensitive one electron transfer process catalyzed by a diaphorase from Clostridium kluyveri. In order to test the hypothesis that O₂ inhibited the transformation of RDX by quenching an electron from RDX, RDX was incubated with diaphorase in the presence of NADH, superoxide dismutase (SOD) and cytochrome c. It was observed that SOD inhibited the reduction of cytochrome c by 37% thus providing evidence for the potential involvement of redox cycling between RDX●⁻ and RDX through which O₂ was converted to O₂●⁻. Control experiments without diaphorase showed that ROX can neither autooxidized nor can it reduce cytochrome c.

C— and N— Mass-Balance and Stoichiometry of the Reaction. Table 2 illustrates carbon and nitrogen mass balance and stoichiometry of reactants consumed and metabolites produced during RDX transformation catalyzed by a diaphorase from Clostridium kluyveri at pH 7.0 and 27° C.

TABLE 2
Metabolites Produced During RDX Transformation Catalyzed by
Diaphorase from Clostridium kluyveri

| | | Concentration | | |
|---|---|---|---|---|
| | | of Reactants/- | | |
| Reactants/ | | metabolites | % Carbon | % Nitrogen |
| Metabolites | | (μmole) | recovery* | recovery* |
| Reactants Consumed | | | | |
| 1. | RDX | 78.0 | 100.0 | 100.0 |
| 2. | NADH | 110.0 | NA | NA |
| Metabolites Produced | | | | |
| 1. | Methylenedinitramine | 9.2 | 4.0 | 8.0 |
| | (MDNA) | | | |
| 2. | Formaldehyde | 207.0 | 88.0 | NA |
| | (HCHO) | | | |
| 3. | Nitrite (NO₂) | 69.0 | NA | 15.0 |
| 4. | Nitrous oxide | 129.0 | NA | 55.0 |
| | (N₂O) | | | |
| 5. | Ammonium ion | 62.0 | NA | 13.0 |
| | +UZ,28/30 (NH₄⁺) | | | |

| | | |
|---|---|---|
| Total % mass recovery | 92.0 | 91.0 |

*Calculated from the total carbon and nitrogen mass in the transformed RDX (78 μmole);
Initial RDX and NADH concentrations were 100 and 150 μmoles, respectively;
NA, not applicable;
Standard deviations are within 6% of the mean absolute values (n = 3).

The reaction stoichiometry (Table 2) indicates that one nitrite molecule was produced per reacted RDX molecule. On the other hand, 78 μmoles of the reacted RDX produced 129 μmoles N₂O and 9.2 μmoles of methylenedinitramine. The latter is unstable in water at pH 7.0 and 27° C. and decomposes quantitatively to produce stoichiometric amounts of N₂O (two molecules) and HCHO (one molecule). Considering that all of the N₂O produced during the reaction comes from the decomposition of methylenedinitramine, it was concluded that one molecule of methylenedinitramine was produced for each reacted RDX molecule. The total nitrogen mass recovery was 91% and was distributed as N₂O (55%), nitrite (15%), ammonium (13%) and methylenedinitramine (8%). The N mass balance data revealed that of the six nitrogen atoms in one RDX molecule, four atoms were finally recovered as nitrous oxide (2 N₂O molecules) whereas the fifth and sixth atoms were present in nitrite (NO₂●) and ammonium (NH₄+), respectively.

The total carbon mass recovery was 92% and it was distributed as formaldehyde (88%) and methylenedinitramine (4%). The latter, as mentioned above, decomposes quantitatively to HCHO and N₂O, indicating that all the three carbon atoms in RDX were finally recovered in the form of HCHO.

Numerically, 1.4 μmoles of NADH were consumed in order to biotransform 1.0 μmole of RDX (Table 2). The stoichiometry of NADH consumption vs RDX transformation supports that 1 molecule of NADH (2e⁻/2H+) was utilized per RDX molecule for its initial denitration. The remaining 0.4 μmoles were either utilized by other transient intermediates and/or end-products following the ring cleavage or bound to protein and thus prevented their detection. The other possibility is that some NADH may be consumed during the futile redox cycling in the presence of traces of oxygen in the reaction medium.

Mechanism of Biotransformation. Without being limiting to the invention in any way, the following mechanism of biotransformation is now put forth for this Example. Based on the oxygen sensitivity, product identification, C— and N— mass-balance, and stoichiometry of the reaction, RDX appears to undergo a mono-denitration process which was responsible. and sufficient for the ring cleavage and secondary decomposition. The absence of nitroso products such as hexahydro-1-nitroso-3,5-dinitro-1,3,5-triazine (MNX), hexahydro-1,3-dinitroso-5-nitro-1,3,5-triazine (DNX) and/or hexahydro-1,3,5-trinitroso-1,3,5-triazine (TNX) also supported the denitration of RDX as the main reaction step responsible for the ring cleavage.

The oxygen inhibition (92%) of RDX transformation and SOD-sensitive cytochrome c reduction experiments suggests that the transfer of a net two redox equivalents (2 e/H+) to the RDX, as determined by stoichiometry of NADH, occurred in a stepwise manner. First step produce RDX●⁻ whose spontaneous denitration would generate nitrite and the free radical RDX. In the second step, RDX● either undergoes H— abstraction to form the amine derivative by acquiring a second redox equivalent (e⁻/H+) or can lose a hydrogen atom to form the cyclohexene derivative.

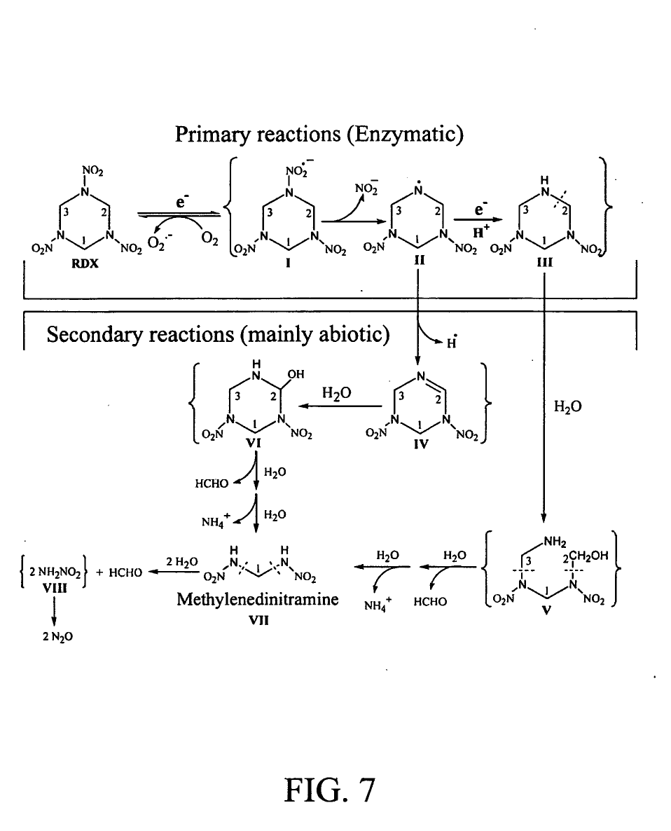

FIG. 7 shows a proposed pathway of diaphorase catalyzed RDX transformation based on stoichiometry of metabolites recovered and NADH consumed. Primary reactions involve RDX reduction to RDX anion radical I that undergoes denitrohydrogenation to produce III. Secondary reactions involve ring cleavage and spontaneous decomposition in water. Intermediates shown inside brackets were not detected.

The stoichiometry of NADH consumption favors the formation of amine derivative (III). The formation of amine derivative (III) via a H-abstraction by the RDX● (II) during photolysis of RDX has been reported previously (Peyton et al., 1999), but no similar biological reactions are known. However, Anusevicius et al. (1998) reported two single e-transfer steps during N-denitration of tetryl catalyzed by a mammalian DT-diaphorase (EC 1.6.99.2).

The proposed pathway in FIG. 7 is consistent with the experimental mass balance of carbon and nitrogen (Table 2). Chapman et al. (1996) reported a similar, but non-enzymatic N-denitrohydrogenation of HMX (a cyclicnitramine explosive) using 1-benzyl-1,4-dihydronicotinamide instead of NADH. However, this reaction needed an initiation by sodium dithionite or light. On the other hand, N-denitrohydrogenation of tetryl using a ferredoxin-NADP+ oxidoreductase (EC 1.1 8.1.2) (Shah et al., 1996) and a mammalian DT-diaphorase (EC 1.6.99.2) (Anusevicius et al., 1993) have also been reported.

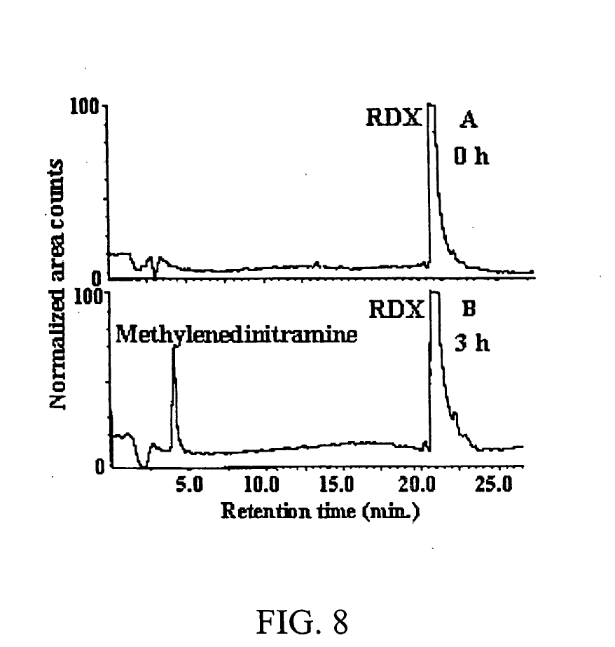

FIG. 8 illustrates LC/MS (ES−) chromatogram of RDX and diaphorase reaction showing methylenedinitramine as a key RDX intermediate. A, 0 hour reaction; B, 3 hour reaction.

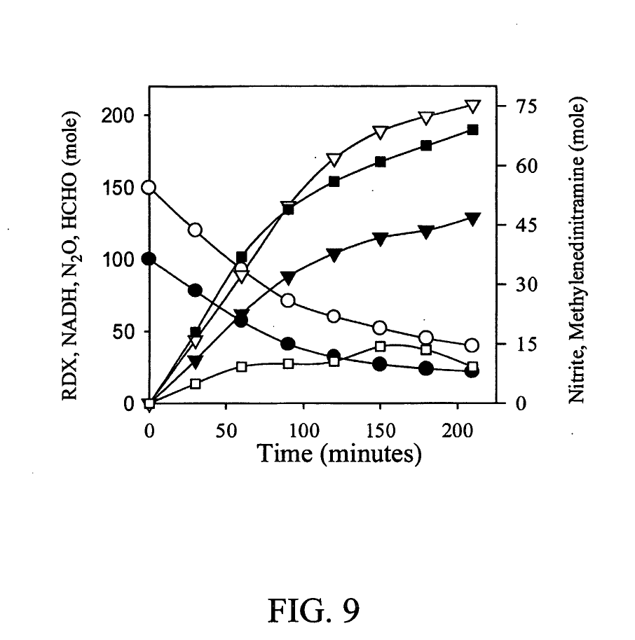

FIG. 9 shows a time course of diaphorase catalyzed RDX transformation with simultaneous production of intermediate and end-products. (●) RDX; (∘) NADH; (▾) N₂O; (∇) HCHO; (▪) Nitrite; (□) Methylenedinitramine. Standard deviations were within 6% of the absolute mean values (n=3).

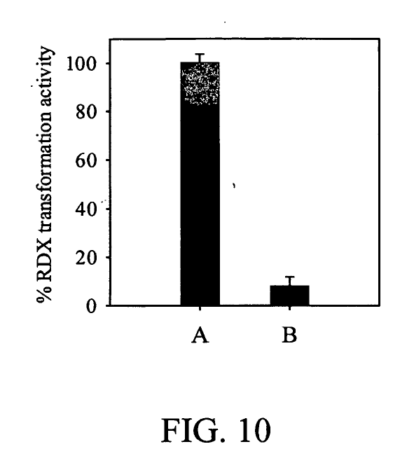

FIG. 10 shows the effect of oxygen on diaphorase catalyzed RDX transformation. A, anaerobic conditions; B, aerobic conditions. 100% RDX transformation activity was equivalent to 47 μmoles RDX transformed h⁻¹mg⁻¹ protein.

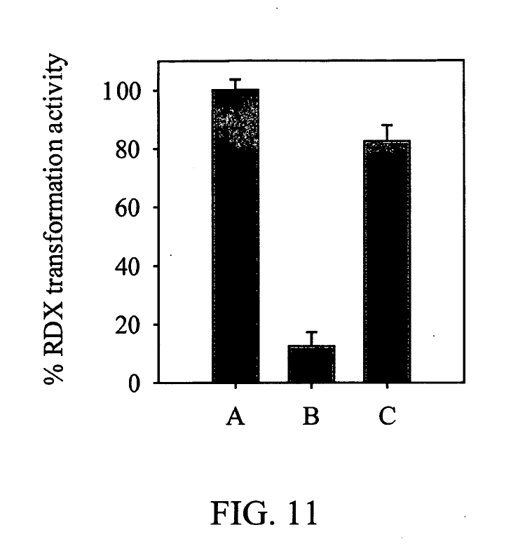

FIG. 11 illustrates the role of FMN in RDX transformation activity of diaphorase. A, native enzyme; B, apoenzyme (deflavo form); C, apoenzyme reconstituted with 200 μM FMN. 100% activity was equivalent to 41 μmoles RDX transformed h⁻¹mg⁻¹ protein.

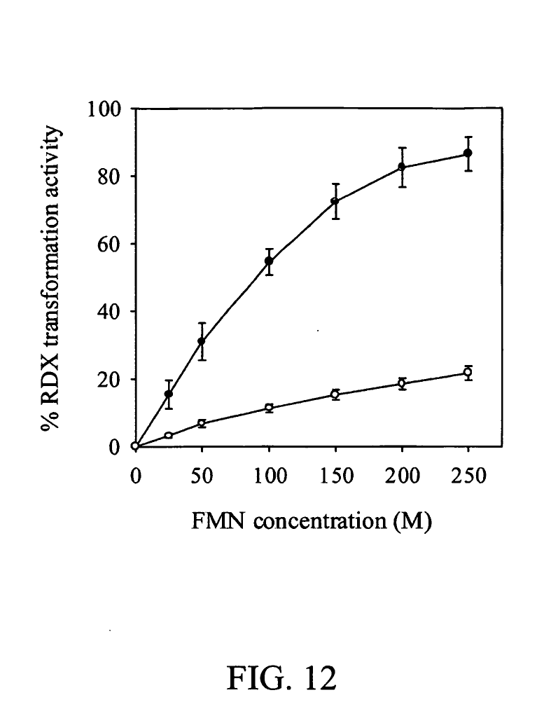

FIG. 12 shows concentration dependent reconstitution of diaphorase by FMN. Symbols: (●) % restored activity of diaphorase; (◯) FMN (commercially available) catalyzed RDX transformation. 100% activity was equivalent to 41 μmoles RDX transformed h⁻¹mg⁻¹ protein.

Involvement of FMN in RDX Transformation. FMN is a redox cofactor of Clostridium kluyveri diaphorase and it is present in the ratio of 1 mole per mole of enzyme (Kaplan et al., 1969). In the present Example, the deflavo enzyme (apoenzyme) lost 87.5% of the RDX transformation activity, suggesting that FMN is an active redox center that possibly mediates the transfer of electrons from NADH to RDX. The remaining 12.5% enzymatic activity may be due to the incomplete removal of FMN from the native enzyme. It was found that when the deflavo enzyme was reconstituted with increasing concentration of FMN, there was a gradual increase in RDX transformation activity up to a maximum of 85% at the FMN concentration of 200 μM. Further, it was found that a commercially available FMN in free form can transform RDX using NADH as electron donor. However, the transformation rate of the free FMN was comparatively lower than the diaphorase-bound FMN. These results indicate that FMN plays an important role in the reduction of RDX by acting as a possible site of electron transfer reaction. In this context, Khan et al. (2002), showed that reduced FMN in pentaerythritol tetranitrate reductase caused the reduction of nitroester explosives (glycerol trinitrate and pentaerythritol tetranitrate) and nitroaromatic explosives (TNT and picric acid). Other reports also showed that the flavin moieties (FAD and FMN) in the flavoenzymes play a key role in substrate reduction Madigan et al., 1993; Lindsay et al., 2000; Xia et al., 2001).

In conclusion, the present Example shows that diaphorase catalyzed N-denitration of RDX is an oxygen-sensitive reaction which possibly requires a net two redox equivalents. Based on this Example, the first redox equivalent appears to lead to the transformation of RDX to RDX●⁻ whose subsequent denitration leads to RDX●. The second redox equivalent possibly causes hydrogenation of RDX leading to the formation of corresponding amine (III). The latter undergoes spontaneous hydrolytic decomposition in water to produce the transient intermediates and end-products. FMN seems to play a key role in transferring the redox equivalents from NADH (donor) to RDX (acceptor).

One of the significant points of the present Example is that Clostridium kluyveri diaphorase catalyzed the RDX transformation at the expense of a net two redox equivalents per RDX molecule which is clearly different and more economical than the earlier reported conventional RDX biotransformation route: via MNX formation (Halasz et al., 2002; Kitts et al., 2000).

### EXAMPLE 3

### Photodegradation of RDX in Aqueous Solution: a Probe for Biodegradation with Rhodococcus sp

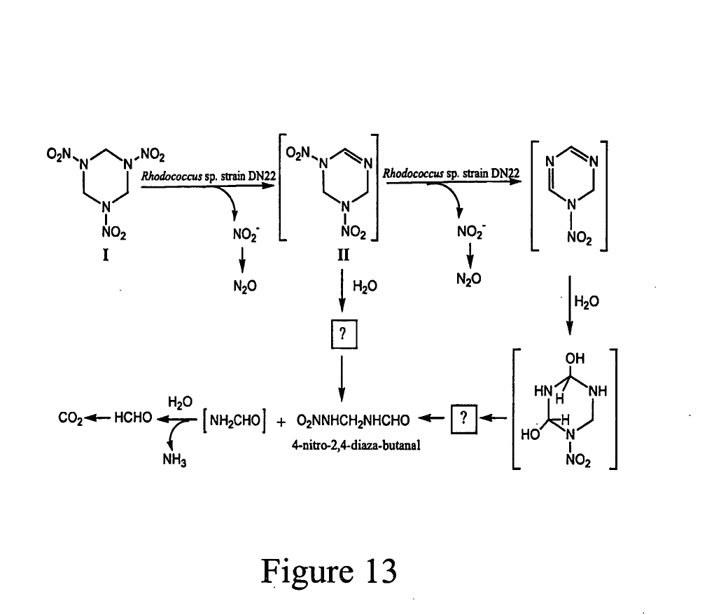

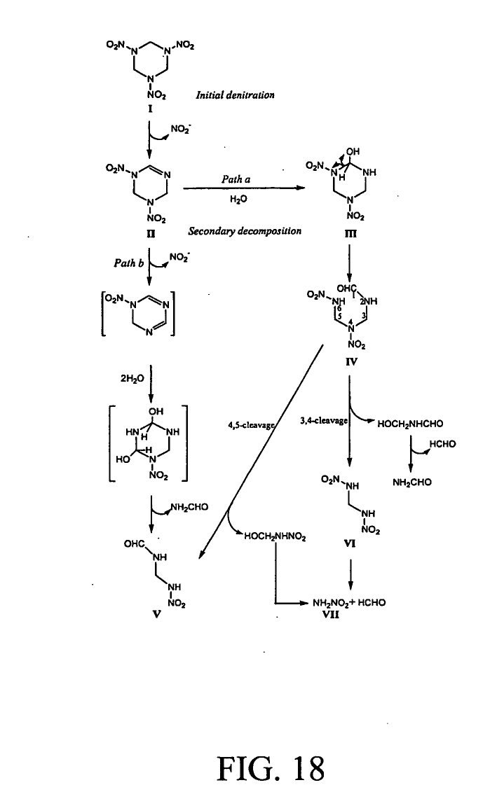

Rhodococcus sp., of a variety of strains, degrades hexahydro-1,3,5-trinitro-1,3,5-triazine (RDX) (1) aerobically via initial denitration followed by ring cleavage. Using UL ¹⁴C-[RDX] and ring labeled ¹⁵N-[RDX] approximately 30% of the energetic chemical mineralized (one C atom) and 64% converted to a dead end product that was tentatively identified as 4-nitro-2,4-diaza-butanal (OHCHNCH₂NHNO₂). To have further insight into the role of initial denitration on RDX decomposition, the energetic chemical was photolyzed at 350 nm and pH 5.5 and monitored the reaction using a combination of analytical techniques. GC/MS-PCI showed a product with a [M+H] at 176 Da matching a molecular formula of C₃H₅N₅O₄ that was tentatively identified as the initially denitrated RDX product hexahydro-3,5-dinitro-1,3,5-triazacyclohex-1-ene (II, shown in FIG. 13 and FIG. 18). Whereas LC/MS (ES−) showed that the removal of RDX was accompanied by the formation of two other key products each showed the same [M−H] at 192 Da matching a molecular formula of C₃H₇N₅O₅ The two products were tentatively identified as the carbinol (III) of the enamine (II) and its ring cleavage product O₂NNHCH₂NNO₂CH₂NHCHO (IV), as shown in FIG. 18. Interestingly, the removal of III and IV was accompanied by the formation and accumulation of OHCHNCH₂NHNO₂ that was detected with strains DN22. At the end of the experiment, which lasted 16 h, the following products were detected: HCHO, HCOOH, NH₂CHO, N₂O, NO₂⁻ and NO₃⁻. Most were also detected during RDX incubation with strain DN22. Finally, no RDX nitroso product was detected during either photolysis and incubation with the aerobic bacteria, emphasizing that initial denitration in both cases was responsible for ring cleavage and subsequent decomposition in water.

Introduction. Both RDX and HMX are toxic and mutagenic and have the tendency to persist in contaminated environment. Rhodococcus strain DN22 can degrade RDX effectively. Other strains show effective degradation, such as Strain A (BRI, Montreal, Canada), strain 11Y (Dr. N. Bruce, Cambridge University, UK). As these various strains of Rhodococcus work under similar conditions as DN22, and yield the same or a similar product distribution, kinetics and stoichiometry as DN22, only strain DN22 is discussed herein in detail. One of skill in the art could easily determine the efficacy of other strains. In this Example, 30% mineralization was achieved, accounting for only one of the three C atoms in RDX leaving the remaining two carbons in a dead end product that was identified as 4-nitro-2,4-diaza-butanal, O₂NNHCH₂NHCHO (FIG. 13). Initial enzymatic denitration of RDX during its incubation with strain DN22 may be responsible for the destruction of the chemical in water. In this Example, the initial denitrated and other early intermediates considered important to the understanding of the degradation process are assessed and characterized.

Since initial enzymatic denitration of RDX was found sufficient to cause spontaneous decomposition, photodenitration of the compound in water should also lead to spontaneous decomposition. Denitration of RDX by photolysis is presumed to be more rapid than enzymatic denitration and therefore might lead to the formation of sufficient amounts of intermediate products to allow detection.

Several studies report the successful photodegradation of RDX via the homolysis of the N—NO₂ bond, under several oxidation conditions. In these cases, photodecomposition of RDX produces common secondary products including HCHO, HCOOH, NH₂CHO, NH₃, NO₂—, NO₃—, and N₂O. Interestingly, most of these secondary photoproducts resembled those observed during aerobic degradation of RDX with strain DN22 (FIG. 13). The resemblance in product distribution between abiotic and biotic degradation of RDX indicates that once the molecule suffers a successful initial attack it undergoes rapid decomposition in water. However, these initial attacks and their resulting intermediate products are not well known.

This Example employs photodenitration of RDX in deionized water at 350 nm in an attempt to generate sufficient amounts of initial intermediate(s) that are not detectable using previous technologies with Rhodococcus sp. strain DN22. The photolysis experiments were conducted in non degassed aqueous solutions at pH 5.5 and room temperature to mimic the conditions employed during RDX degradation with Strain DN22. Low energy wavelength light (350 nm) at neutral conditions was used to minimize the formation of secondary photoproducts whose presence might interfere with other prime products considered important to the understanding of the degradation process. Some of the photolysis experiments were conducted in the presence of acetone in an attempt to enhance denitration through the energetic acetone triplet (T₁(n,π*)) whose excitation energy equals to 326.04 KJmole⁻¹.

Chemicals. Commercial grade RDX (purity >99%) was provided by the Defence Research Establishment Valcartier, Quebec, Canada (DREV). Acetone, formamide and formaldehyde were from Aldrich, Calif. Methylenedinitramine was from the rare chemical department of Aldrich, Canada. MNX with 98% purity was obtained from R. Spanggord (SRI, Menlo Park, Calif.) and TNX (99%) was obtained from G. Ampleman (DRDC, Quebec, Canada). All other chemicals used were of reagent grade.

Irradiation Experiments. A Rayonet photoreactor RPR-100 fitted with a merry-go-round apparatus (Southern New England Co, Hamden, Conn.) equipped with sixteen 350 nm lamps were used as light sources. Photolysis was conducted in 20 ml tubes made of quartz. Each tube was charged with a 10 ml of an aqueous solution of RDX (10 mg/L) in the presence and absence of acetone (250 ppm, v/v). Photolysis was carried out in non degassed quartz tubes sealed with teflon coated mininert valves. Controls containing RDX were kept in the dark during the course of the experiment. The temperature of the reactor was maintained at 25° C. by maintaining the apparatus in a cold room at 10° C. during irradiation. Light intensities, λ 350=3.0 10⁻⁶ einstein ml⁻¹s⁻¹ (RPR-350 nm lamp tubes emitting >90% of their energy at 350 nm) were measured using ferrioxalate actinometry.

Degradation of RDX with Rhodococcus sp. strain DN22. Experimental details on the biodegradation of RDX with strain DN22 can be found in Fournier et al. (2002).

Analytical Procedures. Analysis of 3,5-dinitro- 1,3,5-triaza-cyclohex-1 -ene (II) was carried out on a Hewlett Packard 6890 gas chromatograph coupled to a 5973 quadrupole mass spectrometer in the positive chemical ionization (PCI) mode using methane. Five μl of ethyl acetate extracts of the photolyzed mixture were injected in the solvent vent mode i.e. at 10° C. maintained 0.15 min. then fast heated up to 200° C. under splitless condition on a 15 m×250 um×0.5 um RTX-5amine capillary column from Restek. Helium was used as the carrier gas under an average velocity of 27 cm/sec. The column is heated at 55° C. for 3 minute then raised to 200° C. at a rate of 10° C./min, which was held 2 min. The detector interface was maintained at 200° C. The quadrupole and the source are held at 106 and 150° C., respectively. The product was detected as its protonated molecular mass ion [M+H].

The cyclic carbinol intermediate (I), its ring cleavage product (IV) and their subsequent hydrolyzed products including methylenedinitramine (VI) and 4-nitro-2,4-diaza-butanal (V) were analyzed as described previously using LC/MS (ES−). Briefly, a MicromassTm bench-top single quadrupole mass detector attached to a Hewlett Packard 1100 Series HPLC system and equipped with a photodiode array detector was used. The samples were injected into Supelcosil LC-CN column (4.6 mm ID×25 cm) (Supelco, Oakville, ON) at 35° C. The solvent system consisted of acetonitrile (20%) in water at a flow rate of 1 ml/min. Ionization was done in a negative electrospray ionization mode ES(−) producing mainly the [M−H] mass ions. The mass range was scanned from 40 to 400 Da with a cycle time of 1.6 s and the resolution was set to 1 Da (width at half-height) (Hawari et al., 2000).

Formamide was derivatized with O-(2,3,4,5,6-pentafluorobenzyl)hydroxylamine.HCl (PFBHA) for 30 min. at 80° C., and analyzed by LC/MS (ES−) using a 5 μm Supelcosil LC-8 column (4.6 mm ID×25 cm) (Supelco, Oakville, ON) maintained at 35° C. The solvent system consisted of acetonitrile/water gradient (50 to 90% v/v) at a flow rate of 1 ml/min. Detection was done by monitoring its [M−H] mass ions.

Formic acid, NO₂— and NO₃— were measured using a Hp3D CE instrument model 1600 equipped with a diode array detector. The system was fitted with a Agilent G16006132 fused silica capillary with a total length of 64.5 cm (56 cm effective) and an internal diameter of 50 μm. The voltage was set at 25 kV (negative polarity) and the temperature at 25° C. Samples were injected by applying 50 mbar pressure to the capillary inlet for 50 s. The electrolyte solution was prefiltered and buffered with triethanolamine at pH 7.7. Hexamethonium hydroxide is added as a cation flow modifier. Separation time was 15 minutes. Indirect detection at 254 nm was used with pyromellitic acid as the background absorbing ion. Detection limit was 50 ppb.

Ammonium cation was analyzed using an SP 8100 HPLC system equipped with a Waters 431 conductivity detector and a Hamilton PRP-X200 (250 mm×4.6 mm×10 μm) analytical cation exchange column as described by Hawari et al. (2001). For trace analysis concentration of NH₄⁺ was measured by capillary electrophoresis using a HP3D CE instrument model 1600 equipped with a diode array detector. The system was fitted with a Agilent G16006132 fused silica capillary with a total length of 64.5 cm (56 cm effective) and an internal diameter of 50 um. The voltage was set at 15 kV (positive polarity) and the temperature at 25° C. Samples were injected by applying 50 mbar pressure to the capillary inlet for 50 s. The electrolyte solution was prefiltered and buffered with lactic acid at pH 3.5. 18-crown 6 is added as a anion flow modifier. Separation time was 15 minutes. Indirect detection at 350 nm was used with imidazole as the background absorbing ion. Detection limit was 50 ppb.

Analysis of N₂O was carried out with a GC/ECD system as previously described (Hawari et al., 2000). Formaldehyde (HCHO) was analyzed as described by Summers (1990) with minor modifications. Samples were derivatized with 2,4-pentanedione in the presence of ammonium acetate and glacial acetic acid for one hour at pH 6.0, 40° C. The derivatives were then analyzed by HPLC using a 5 μm Supelcosil LC-8 column (4.6 mm ID×25 cm) (Supelco, Oakville, ON) maintained at 40° C. The mobile phase consisted of an acetonitrile gradient of 15% to 27%, at a flow rate of 1.5 ml/min for 6 min. Detection and quantification were carried out using a fluorescence detector (excitation at 430 nm and emission at 520 nm). Measurement of methanol is made on a Hewlett Packard 6890 gas chromatograph coupled to an FID. 1 μL of water sample is injected on a 2 m×0.03 mm Hayesep Q micropacked column from Supelco. The column is heated at 60° C. one minute then raised to 180° C. at a rate of 20° C./minute. Helium is used as carrier gas. The injector and detector are maintained at 150° C. and 250° C. respectively. Standards are prepared from neat compounds from JT Baker. Detection limit is 0.25 ppm.

Results and Discussion

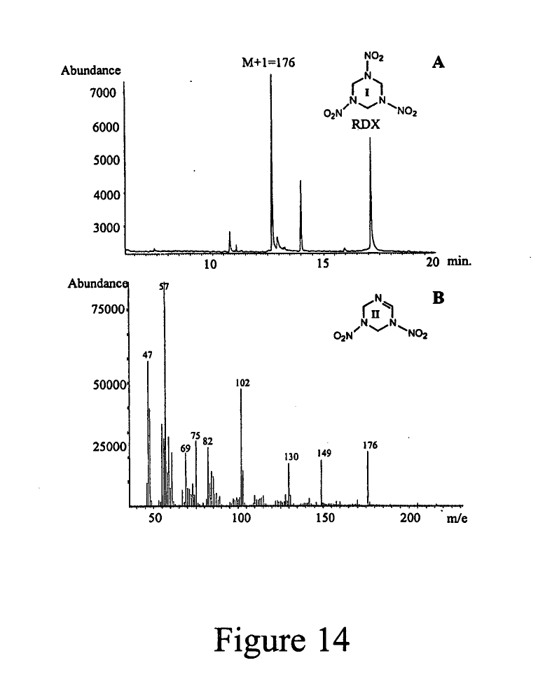

Photodegradation of RDX in aqueous solution at 350 nm. FIG. 14 part A is a typical GC/MS (PCI) chromatogram of RDX after 2 h of photolysis at 350 nm showing a product with a retention time at 12.7 min and a protonated molecular mass ion [M+H] at 176 Da, matching a molecular formula of C₃H₅N₅O₄. The product also showed several other characteristic mass ions at 47, 102 and 149 Da representing —HNO₂, CH₂NNO₂CH₂N and O₂NHCH₂NNO₂CH₂N, respectively (FIG. 14 part B). The mass data of II matched exactly those of an earlier intermediate that was tentatively identified as hexahydro-3,5-dinitro-1,3,5-triaza-cyclohex-1-ene that we detected during hydrolysis of RDX in alkaline 2-propanol (Hawari et al., 1996).

The present RDX photo product was tentatively identified as hexahydro-3,5-dinitro-1,3,5-triazacyclohex-1-ene. However, when a standard material of RDX was injected on the same GC, a peak was observed that had the same r.t. and [M+H] as that obtained during RDX photolysis. RDX is thermally unstable and decomposes via HNO₂ elimination (Zhao et al., 1988), indicating that the energetic chemical might also undergo thermal decomposition at least partially during GC analysis. However, the GC area of II generated during RDX photolysis was found to be approximately twice as large as the area obtained from dark controls, suggesting its potential formation as a photo product. Previous reports indicated the formation of II during photolysis, thermolysis and hydrolysis of RDX, but product II has never been isolated. Product II is an enamine with a reactive —C═N bond that is expected to undergo rapid hydrolysis in water more likely to be unstable in water.

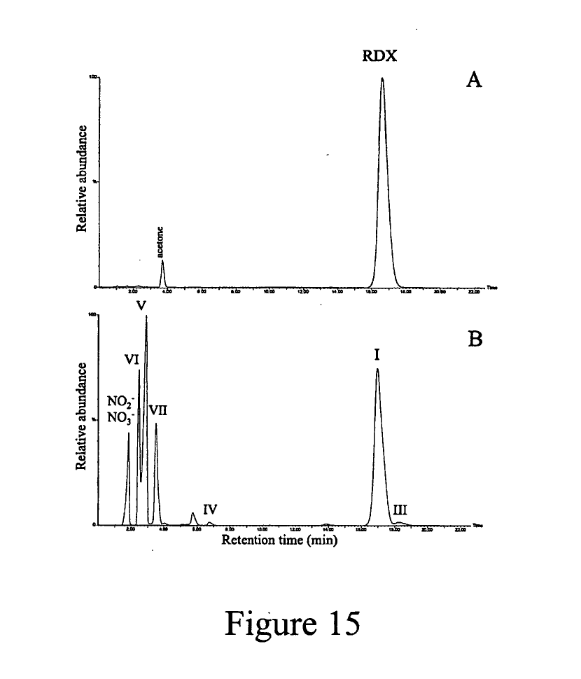

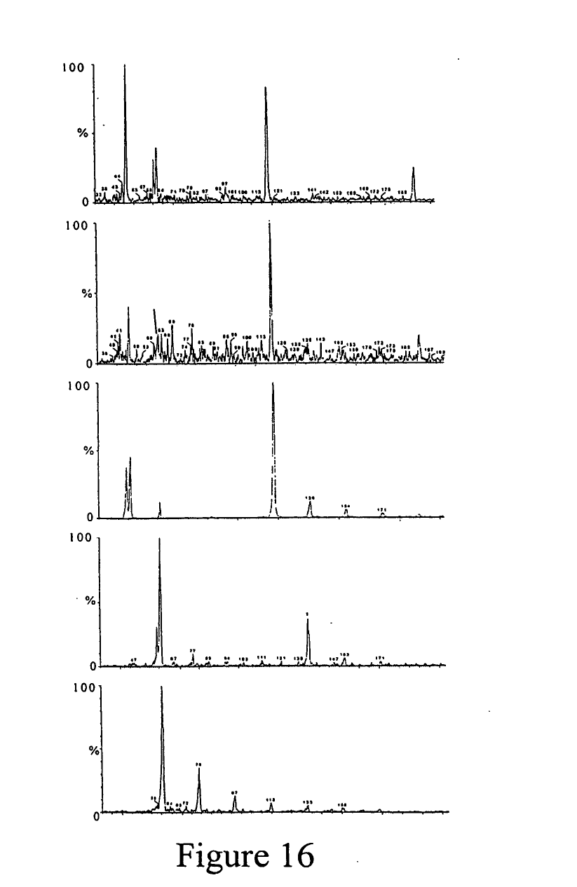

When photolysis was carried out in the presence of acetone, two new LC/MS peaks marked III and IV were detected at 6.8 and 18.2 min respectively as shown in FIG. 15, part B. Each showed the same [M−H] at 192 Da, matching a molecular formula of C₃H₇N₅O₅ (MW 193 Da). The two products also showed several other characteristic mass ions at 46, 61 and 118 Da, representing —NO₂, —NHNO₂ and —OHCNCH₂NHNO₂ (FIG. 16). Peak III, which appears next to RDX at a higher r.t. was presumed to be less polar and thus was tentatively identified as the cyclic carbinol pentahydro-2,4-dinitro-2,4,6-triaza-cyclohexanol (M) derivative of II. Whereas peak IV, which appears at a lower r.t was tentatively identified as the ring cleaved product 6-nitro-2,4,6-triaza-hexanal (O₂NNHCH₂NNO₂CH₂NHCHO) of III (FIG. 16). In the absence of acetone peaks, products III and IV were only detected in trace amounts.

Two other familiar intermediates, V at a r.t of 2.9 min with a [M−H] at 118 Da, matching a molecular formula of C₂H₅N₃O₃ and VI at a r. t. of 2.3 min with a [M−H] at 135 Da matching a molecular formula of CH₄N₄O₄ were observed (FIG. 15 and FIG. 16). V was identified as 4-nitro-2,4-diaza-butanal by comparing its chromatographic and mass data with those of a standard material of OHCNHCH₂NHNO₂ obtained by incubating RDX with strain DN22 (5). Whereas VI was identified as methylenedinitramine, O₂NNHCH₂NHNO₂, by comparison with a commercial reference standard.

Interestingly, the LC/MS(ES−) spectrum of IV was found to contain a strong mass ion at 118 Da (FIG. 16), suggesting that this intermediate may have acted as a precursor to O₂NNHCH₂NHCHO (V) whose [M−H] was also observed at 118 Da. Consequently, the dominant formation of V (64%) during biodegradation of RDX with Rhodococcus indicates the direct involvement of intermediate IV in its formation too. The last LC/MS (ES−) peak VII appearing at 3.9 min showed mainly a strong mass ion at 61 Da and was tentatively identified as the nitramide H₂NNO₂ (VI) (FIG. 16). Spontaneous decomposition of VII in water produces nitrous oxide, N₂O (Hawari, 2000).

The present mild photolysis (350 nm) of RDX in non degassed aqueous solution did not produce any of the RDX nitroso derivatives such as MNX, DNX and TNX. Interestingly none of the nitroso products was detected during RDX aerobic degradation with strain DN22. Fenton reagent (Fe⁺²/H₂O₂), which is known to be a powerful generator of the highly reactive hydroxyl radicals, OH, mineralized both RDX and HMX to CO₂ and NO₃— without producing the nitroso compounds (Zoh et al., 2000). However, photolysis of RDX at short wavelengths (higher energy) produced the nitroso derivatives (Peyton et al., 1999). Nitroso products were frequently detected during biodegradation of RDX under anaerobic conditions. Interestingly when RDX was photolyzed under a blanket of argon (anaerobic conditions) at shorter wavelengths (254 nm) all three nitroso products were detected.

At the end of photolysis, which lasted 16 h the accumulation of the products NO₂⁻, NO₃⁻, NH₂CHO, HCHO, HCOOH, N₂O, NO₂NHCH₂NHCHO and NO₂NHCH₂NHNO₂ was observed. Most of these products were also detected during RDX incubation with the aerobic degrader DN22, emphasizing that once a successful initial attack takes place on RDX the molecule undergoes spontaneous decomposition in water.

The presence of acetone during RDX photolysis did not seem to drastically change product distribution. For instance, all products that were detected during RDX photolysis in the absence of acetone were also detected during its presence. Some slight variation in the relative distribution of these products was noted. Preliminary investigation with other photo sensitizes such, as phenothiazine and benzyl viologen also did not seem to drastically affect RDX product distribution. No RDX degradation was observed in dark aqueous controls with and without the sensitizer.

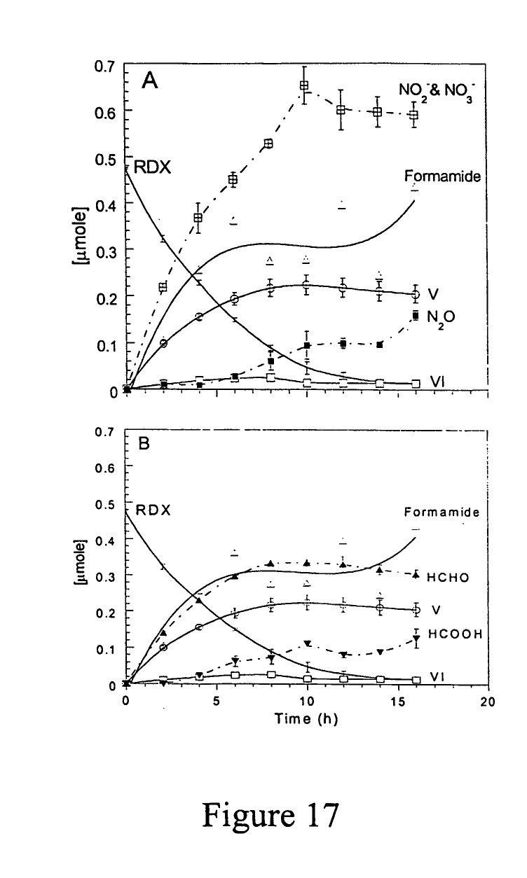

Kinetics and Stoichiometry—A Comparative Study with Strain DN22. The removal of RDX during photolysis was accompanied by the concurrent formation of nitrite NO₂⁻ and nitrate NO₃⁻ ions (FIG. 17 part A). At the end of the experiment, which lasted 16 h almost all of the NO₂⁻ transformed to NO₃⁻, suggesting that nitrate ion was produced from nitrite photo-oxidation. For instance when NaNO₂ was photolyzed at 350 nm under the same conditions used to photolyze RDX, NO₃⁻ was detected. Bose et al. (1998) reported that photo-oxidation of RDX with ozone at 230 nm also produced nitrate. In contrast incubation of RDX with Rhodococcus strain DN22 produced NO₂⁻ without nitrate ion. The microorganism initiated enzymatic denitration to get its own need of nitrogen for growth since RDX was used as the sole source for nitrogen.

Photodegradation of RDX was also accompanied by the accumulation of NH₂CHO, HCHO, and HCOOH (FIG. 17 part B). In the previous RDX biodegradation study with Rhodococcus, HCHO was detected which latter mineralized (liberated as ¹⁴CO₂), but NH₂CHO was inferred to exist by the detection of its degradation products ammonia and HCHO. For instance, when NH₂CHO was incubated with strain DN22 the amide degraded to ammonia and HCHO.

FIG. 17 part A also showed that the removal of RDX was accompanied by the accumulation of the ring cleavage product 4-nitro-2,4-diaza-butanal (V), which was also detected during RDX biodegradation with DN22. However, V was found to persist indefinitely in incubation mixtures with strain DN22 at pH 7, but degraded slowly with light.

Table 3 shows carbon and nitrogen mass balances of RDX (0.46 μmole) after 16 h of photolysis at 350 nm in water. These data illustrate that the N mass balance of RDX removal is 73% distributed as follows O₂NNHCH₂NHCHO (V) (22.2%), NO₂⁻ and NO₃⁻ (21.4%), NH₂CHO (15.7), N₂O (11.6%) and CH₂(NHNO₂)₂ (VI) (1.9%) and the C mass balance is 93% distributed as follows: NH₂CHO (31.9%), O₂NNHCH₂NHCHO (V) (31.5%), HCHO (22.0%), HCOOH (9.1%), and CH₂(NHNO₂)₂ (VI) (0.9%). The nitrogen content of nitramide (NH₂NO₂) is not included in the N mass balance because of the absence of a standard material for quantification. In comparison, N— and C— mass balances of RDX biodegradation with strain DN22 were calculated as 90 and 94%, respectively. In the latter case approximately 30% of the C-content (1 atom) of RDX were found in ¹⁴CO₂ and 64% (2 C atoms) were incorporated in the dead end product V. The slight differences in product distribution and N and C mass balances between photolysis and biodegradation experiments was possibly caused by some variations in the relative reactivities of certain intermediates toward light and enzymes.

TABLE 3
Carbon and Nitrogen mass balances of RDX Photolysis at 350 nm

| Products | μmole ± SD | C-content % | N-content % |
|---|---|---|---|
| O₂NNHCH₂NHCHO(V) | 0.205 ± 0.019 | 29.6 | 22.2 |
| H₂NCHO | 0.436 (a) | 31.5 | 15.7 |
| CH₂(NHNO₂)₂ (VI) | 0.013 (a) | 0.9 | 1.9 |
| NO₂⁻ + NO₃⁻ | 0.591 ± 0.027 | — | 21.4 |
| N₂O | 0.160 ± 0.009 | — | 11.6 |
| HCHO | 0.304 ± 0.011 | 22.0 | — |
| HCOOH | 0.126 ± 0.026 | 9.1 | — |
| % mass balance | | 93.1 | 72.8 |

(a) indicates a single measurement.

Insights into Photodecomposition Mechanism of RDX: Parallels with Strain DN22: Initial Denitration. FIG. 17 part A shows that the removal of RDX was concurrent with the formation of nitrite, NO₂⁻ (and NO₃⁻), indicating the occurrence of an important initial denitration step during degradation. The absence of any of RDX nitroso products MNX, DNX and TNX supported our belief that initial denitration was responsible for RDX subsequent degradation. Likewise, initial enzymatic denitration of RDX with Rhodococcus sp. strain DN22 was found sufficient to cause ring cleavage and subsequent decomposition in water.

During RDX photolysis in the presence of acetone the ketone should produce a triplet T₁(n,π*) with excitation energy equals to 326.0 KJmole⁻¹ (Turro 1978). Subsequent hydrogen atom abstraction from one of RDX methylene groups by acetone T₁(n,π*) would generate an RDX radical RDX●. Elimination of NO₂ from RDX● should lead to the formation of the enamine II. This reaction is exothermic because the bond dissociation energies (BDE) of the cleaved C—H (in RDX) and the generated O—H (in acetone) bonds are 376.2 and 418,0 KJmole⁻¹, respectively. However, the energy associated with acetone triplet T₁(n,π*) is high (326.0 KJmole⁻¹) and should be sufficient to cleave the N—NO₂ bond (BDE 204.9 KJmole⁻¹). Alternatively, direct homolytic cleavage of the N—NO₂ bond in RDX should not be excluded. For instance the energy associated with λ 350 nm is 342 KJmole⁻¹ and therefore should be sufficient to cleave the N—NO₂ bond (BDE 204.9 KJmole⁻¹) (Behrens et al., 1991). However, a synchronous bimolecular elimination (E₂) of one molecule of HNO₂ during RDX photolysis has been suggested to occur (Peyton et al., 1999; Melius et al., 1990). Nonetheless such elimination would also lead to the formation of the enamine II (FIG. 18). As mentioned previously, the presence and absence of sensitizers such as acetone, phenothiazine and benzyl viologen during RDX photolysis did not seem to change product distribution drastically. Therefore it is difficult to determine which among the previous denitration routes was the most probable one.

Secondary Decomposition. The potential involvement of the enamine II in the initial decomposition of RDX is supported by our observation of its carbinol derivative (III) and its subsequent ring cleavage product (IV) (FIG. 18). As we mentioned previously enamines are unstable in water and therefore II should react through its —C═N bond with an H₂O molecule to produce the carbinol III. The latter is also expected to be unstable and should undergo rapid ring cleavage across its NNO₂—CH(OH) to produce IV (FIG. 18). For instance, α-hydroxylation of dialkylnitrosamine by mixed function oxidase produces unstable carbinol products that tend to cleave easily at the C(OH)-NNO₂ bond in water.

Intermediate IV with several labile C—NNO₂ bonds is thus expected to decompose in water to give several products depending on which bond is hydrolyzed first. For example 4,5-cleavage produces 4-nitro-2,4,-diaza-butanal (V) whereas 3,4 cleavage produces methylenedinitramine (VI) (FIG. 18). Although we were unable to detect III and IV during RDX incubation with Rhodococcus, this Example suggests their presence based on the predominant formation of their hydrolyzed product V (FIG. 18). A high denitration rate should result in the formation of sufficient amounts of these early intermediates to permit detection. Subsequent reactions of these early intermediates in water is expected to be rapid and thus their product distribution will not be drastically influenced by light or enzyme. After 16 h of photolysis it was found that the disappearance of 0.46 μmole of RDX produced approximately 0.6 μmole of nitrite and nitrate ions, indicating that for every reacting molecule of RDX there was more than one N atom but less than two that were involved in the formation of nitrite (and nitrate) ion. Therefore the involvement of two denitration routes in the degradation of RDX are suggested; a mono denitration route leading to the formation of II (FIG. 18, path a) and a second route which involved two denitration events prior to RDX ring cleavage (FIG. 18, path b). In FIG. 18, path a leads to the detected intermediates IV, V, VI and VII whereas path b leads to V. During RDX incubation with DN22 resting cells, there were two nitrite ions produced for each disappearing molecule of RDX, suggesting the involvement of the cyclic dicarbinol intermediate prior to ring cleavage (FIG. 18, path b). Thus, potential involvement of II, III an IV as key intermediates during RDX incubation with DN22 is supported by the direct observation of these intermediates during photodenitration of the energetic chemical.

In conclusion, FIG. 18 represents the best explanation for the detected RDX degradation products obtained during photolysis and incubation of the chemical with Rhodococcus sp. DN22. FIG. 18 clearly shows that the reaction steps involved in the photodecomposition of RDX are replicates of those occurring during biodegradation of the energetic chemical under aerobic conditions with strain DN22. These findings suggest that successful initial enzymatic or chemical attack, denitration in the present study, occurs on RDX (or HMX), the energetic chemical will decompose rapidly in water.

### EXAMPLE 4

### Alkaline Hydrolysis of RDX, HMX and CL-20

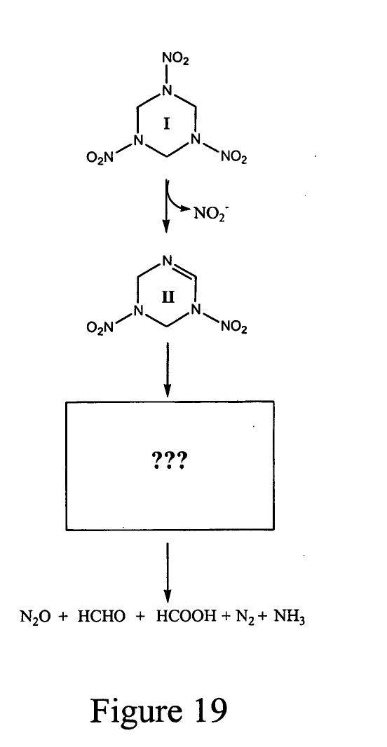

Roman numeral reference indicia used in the Example pertain to FIGS. 19, 25 and 26, as appropriate.

Hexahydro-1,3,5-trinitro-1,3,5-triazine (RDX, I), and octahydro-1,3,5,7-tetranitro-1,3,5,7-tetrazocine (HMX), are known to hydrolyze at pH>10 to produce end products including NO₂⁻, HCHO, HCOOH, NH₃, and N₂O, but little information is available on their precursors apart from the tentatively identified pentahydro-3,5-dinitro-1,3,5-triaza-1-ene (II). In the present study, we hydrolyzed RDX, MNX (hexahydro-1-nitroso-3,5-dinitro-1,3,5-triazine) and HMX in aqueous solution (pH 10-12.3) and found that the removal of the nitramine was accompanied by the formation of one molar equivalent of nitrite and the accumulation of the key ring cleavage product 4-nitro-2,4-diaza-butanal (4-NDAB, O₂NNHCH₂NHCHO). Most of the remaining C and N content of RDX, MNX and HMX was found in NO₂⁻, HCHO, N₂O, HCOOH, and NH₃. Consequently, we selected RDX as a model cyclic nitramine compound and hydrolyzed it in aqueous acetonitrile solutions (pH 10-12.3) in the presence and absence of hydroxypropyl-β-cyclodextrin (HP-β-CD) to explore other early intermediates in more detail. We observed a transient LC-MS peak with a [M−H] at 192 Da that was tentatively identified as 4,6-dinitro-2,4,6-triaza-hexanal (O₂NNHCH₂NNO₂CH₂NHCHO, III) considered as the hydrolyzed product of II. In addition we detected another novel intermediate with a [M−H] at 148 Da that was tentatively identified as a hydrolyzed product of III, namely, 5-hydroxy-4-nitro-2,4-diaza-pentanal (HOCH₂NNO₂CH₂NHCHO, IV). Both III and IV can act as precursors to 4-NDAB. The results provide strong evidence that initial denitration of cyclic nitramines in water is sufficient to cause ring cleavage followed by spontaneous decomposition to form the key product 4-NDAB. Finally, in the case of 2,4,6,8,10,12-hexanitro-2,4,6,8,10,12-hexaazaisowurtzitane (CL-20), denitration (two NO₂⁻) also led to the formation of HCOOH, NH₃, and N₂O, but neither HCHO nor 4-NDAB were detected.

Introduction. The cyclic nitramines hexahydro-1,3,5-trinitro-1,3,5-triazine (RDX) and octahydro-1,3,5,7-tetranitro-1,3,5,7-tetrazocine (HMX) are highly energetic compounds used in various propellants and conventional munitions (FIG. 1). The manufacturing and usage of these toxic munitions has resulted in severe contamination of both soils and groundwater, thus necessitating their safe removal from the environment.

Numerous investigations have established that the typical decomposition or degradative routes (photochemical, thermal, microbiological, or hydrolytic) of RDX produces end products including nitrite (NO₂⁻), nitrous oxide (N₂O), N₂, ammonia (NH₃), formaldehyde (HCHO), formic acid (HCOOH) and CO₂. Hoffsommer et al., (1997) reported that RDX degradation in alkaline medium was initiated by a single denitration step to first form pentahydro-3,5-dinitro-1,3,5-triazacyclohex-1-ene (II), which then hydrolyzed further leading to rapid ring cleavage and decomposition (FIG. 19). While only a few studies have been conducted with HMX, it has been shown that HMX undergoes alkaline hydrolysis at a slower rate than RDX, to give a product distribution (NO₂⁻, N₂O, NH₃, N₂ and HCOOH) similar to that obtained with RDX. In the case of the polycyclic nitramine 2,4,6,8,10,12-hexanitro-2,4,6,8,10,12-hexaazaisowurtzitane (CL-20), the compound has only recently been synthesized and is a more powerful explosive than either RDX or HMX. Therefore, little information is currently available on its chemical and biological activity and fate.

Both Rhodococcus sp. strain DN22 and light (350 nm) can successfully decompose RDX in water via initial denitration to produce 4-nitro-2,4-diazabutanal (4-NDAB, O₂NNHCH₂NHCHO) as a major ring cleavage product in both cases. In addition the end products found in photochemical and enzymatic degradative routes are basically similar and include NO₂⁻, N₂O, NH₃, HCHO, CO₂, and H₂NCHO.

While many of the end products of RDX hydrolysis are known, intermediates and decomposition pathways are not well understood. Heilmann et al. (1996) suggested that RDX and HMX contaminated waters could be economically treated using granular activated carbon (GAC) to first adsorb the explosive, followed by off-line regeneration of the laden GAC through alkaline hydrolysis. However, for this technology to be optimized, knowledge of intermediates and the degradation pathways of these two monocyclic nitramines is critical both to predict their behavior and enhance their removal from the environment.

The end products of RDX decomposition during enzymatic biodegradation with strain DN22 were similar to those obtained via both alkaline hydrolysis and photolysis. This Example investigates whether following initial denitration of the cyclic nitramine, ring cleavage and subsequent decomposition are largely dictated by the aqueous chemistry of the resulting intermediates. Therefore, if other cyclic nitramines such as MNX (hexahydro-1-nitroso-3,5-dinitro-1,3,5-triazine), HMX, and CL-20 produce end products similar to those of RDX, then their decomposition mechanisms should also be similar. Hence, this Example provides additional insight into the degradation pathways of these cyclic nitramines by investigating the initial steps and intermediates that are involved in their decomposition under alkaline conditions. RDX was selected as a model cyclic nitramine compound and hydrolyzed it in aqueous acetonitrile solutions (pH 10-12.3) in the presence and absence of hydroxypropyl-β-cyclodextrin (HP-β-CD) in an attempt to explore other early intermediates in more detail.

Chemicals. Commercial grade RDX (purity >99%), ring-labelled [¹⁵N]-RDX (purity ca. 98%), HMX (purity>99%), and hexahydro-1,3,5-trinitroso-1,3,5-triazine (TNX, 99% pure) were provided by the Defense Research and Development Canada (DRDC), Valcartier, Canada. 2,4,6,8,10,12-hexanitro-2,4,6,8,10,12-hexaazaisowurtzitane (ε-CL-20) was provided by A.T.K. Thiokol Propulsion (Brigham City, Utah). Hexahydro-1-nitroso-3,5-dinitro-1,3,5-triazine (MNX, 99% pure) was provided by R. Spanggord (SRI, Menlo Park, Calif.). 4-nitro-2,4-diazabutanal was obtained as described by Fournier et al. (2002), and methylene dinitramine was obtained from the rare chemical department, Aldrich, Canada. All other chemicals used in this work were of reagent grade.

Hydrolyses of RDX, MNX, HMX and CL-20 in Aqueous Solution at pH 10. To a series of dry 20 mL vials (each wrapped in Aluminum foil) was added an aliquot of cyclic nitramine stock solution prepared in acetone. The acetone was evaporated in a fume hood, followed by the addition of 10 mL of a NaOH solution (pH 10) to give the following concentrations 215, 180, 110 and 100 ppm for RDX, MNX, HMX and CL-20, respectively. The initial concentrations were kept well in excess of the maximum water solubility of each nitramine (ca. 50 ppm for RDX, 5 ppm for HMX, and 3.6 ppm for CL-20) in an attempt to generate sufficient amounts of intermediates to allow detection. The vials were sealed with Teflon coated serum caps and placed in a thermostated benchtop shaker at 30° C. and 200 rpm. Some vials were sparged with Argon for 2 hours to allow analysis for N₂.

We used a gas-tight syringe to measure gaseous products (N₂O and N₂) in the head space but for the analysis of the liquid medium we first quenched the reaction by adding HCl taken in a MeCN solution (10 mL) (pH 4.5). MeCN was used to solubilize unreacted starting material for subsequent analysis. The quenched vials were then stored in a refrigerator at 4° C. until analyzed.

Hydrolysis of RDX in 70% MeCN:30 H₂O (v/v) Mixture. A predetermined weight of RDX (0.1 g) was placed into a vial to which a 10 mL solution of NaOH (pH 12.3) in 70:30 (v/v) acetonitrile: water was added, giving an initial RDX concentration of 10,000 ppm. MeCN was used to solubilize the high RDX concentration used, while a high pH was used to accelerate the reaction. The vial was sealed with a Teflon coated serum cap and placed in a thermostated benchtop shaker at 30° C. and 200 rpm. After 22 h, the reaction was quenched by the addition of HCl (1 M) and the volume of the quenched mixture was then reduced under reduced pressure (18 mm Hg). The remaining mixture was passed through a column containing 3-aminopropyl-functionalized silica gel (Aldrich) using a 70% acetonitrile:30% water (v/v) mixture to remove chloride and nitrite anions, preventing interference during LC-MS (ES−) analysis (discussed below). This experiment was then repeated using [¹⁵N]-RDX.

Hydrolysis of RDX in the Presence of Hydroxypropyl-β-Cyclodextrin (HP-β-CD). HP-β-CD was used in an effort to stabilize putative early intermediates such as pentahydro-3,5-dinitro-1,3,5-triazacyclohex-1-ene (II) and its ring cleavage products (O₂NNHCH₂NNO₂CH₂NHCHO (III)) and HOCH₂NNO₂CH₂NHCHO (IV) by forming inclusion complexes. RDX (0.1 g) or [¹⁵N]-RDX (0.1 g) was placed in a vial, followed by the addition of 10 mL NaOH (pH 12.3) in 65:35 (v/v) acetonitrile:water containing 3% (wt/v) HP-β-CD. Hydrolysis and sample preparation for subsequent analysis was conducted as discussed above.

Analytical Procedures. The concentration of RDX, MNX, HMX and CL-20 was determined using a reversed-phase HPLC connected to a photodiode array (PDA) detector. Formaldehyde (HCHO), ¹⁵N¹⁴NO (45 Da) and ¹⁴N¹⁴NO (44 Da) analyses were carried out as described by Sheremata et al. (2000). Analyses of nitrite (NO₂⁻), nitrate (NO₃⁻), fornate (HCOO⁻) and ammonium (NH₄⁺) were performed by capillary electrophoresis on a Hewlett-Packard 3D-CE equipped with a Model 1600 photodiode array detector and a HP capillary part number 1600-61232. The total capillary length was 64.5 cm, with an effective length of 56 cm and an internal diameter of 50 μm. For nitrite, nitrate and formate, analyses were performed using sodium borate (25 mM) and hexamethonium bromide (25 mM) electrolytic solution at pH 9.2. The ammonium cation was analyzed using a formic acid (5 mM), imidazole (10 mM) and 18-crown-6 (50 mM) electrolytic solution at pH 5. In all cases, UV detection was performed at 215 nm.

A Micromass™ bench-top single quadropole mass detector attached to a Hewlett Packard 1100 Series HPLC system equipped with a DAD detector was used to analyze for RDX ring cleavage intermediates. The samples were injected into a 4 μm-pore size Supelcosil CN column (4.6 mm ID by 25 cm; Phenomenex, Torrance, Calif.) at 35° C. The solvent system was composed of 20% acetonitrile: 80% water (v/v): For mass analysis, ionization was performed in a negative electrospray ionization mode, ES(−), producing mainly the deprotonated molecular mass ions [M−H]. The mass range was scanned from 30 to 400 Da with a cycle time of 1.6 s, and the resolution was set to 1 Da (width at half height).

Results and Discussion

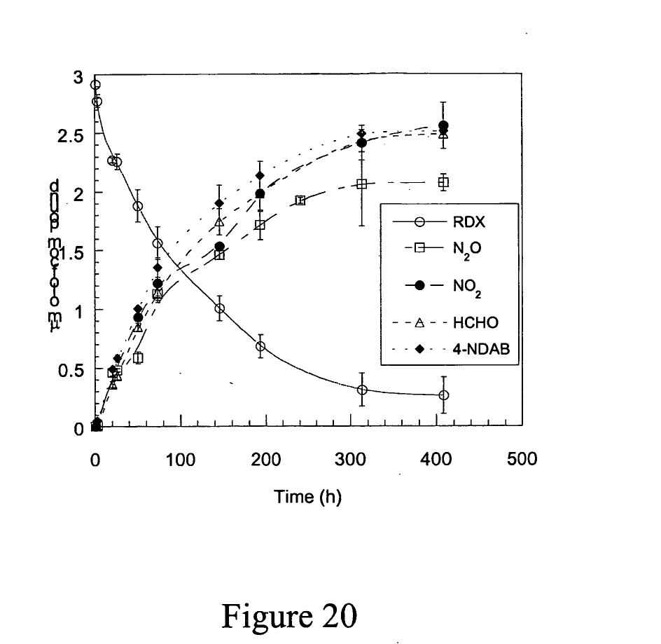

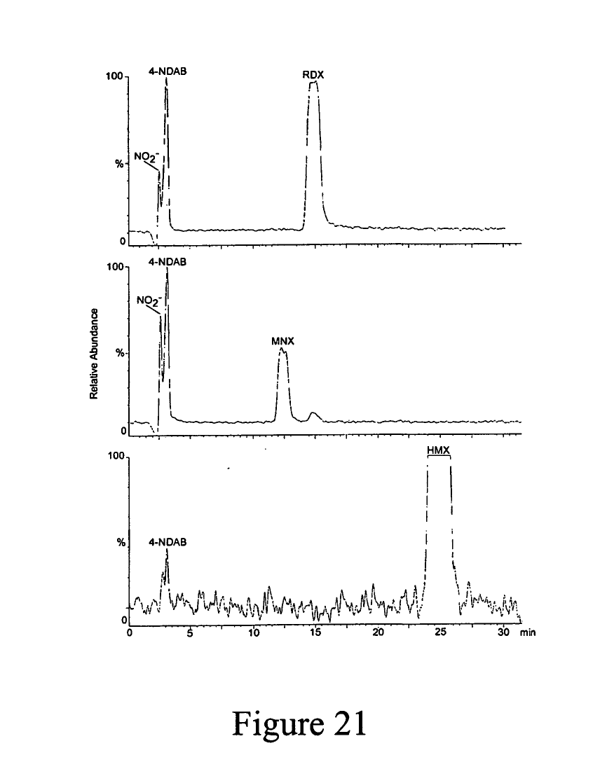

Alkaline Hydrolysis of Cyclic Nitramines: Product Distribution. FIG. 20 shows that disappearance of RDX during hydrolysis in water (pH 10) is concomitant with the formation and accumulation of NO₂—, N₂O and formaldehyde (HCHO). Although we were unable to detect formamide, we detected its hydrolyzed products HCOO⁻ (0.18 μmole) and NH₄⁺ (0.20 μmole). Amides are known to undergo spontaneous hydrolysis under alkaline conditions to produce the corresponding amines and acids. FIG. 20 also shows that the disappearance of RDX was accompanied by the accumulation of the ring cleavage product 4-nitro-2,4-diaza-butanal (4-NDAB,O₂NNHCH₂NHCHO). 4-NDAB was also a key product during photo denitration and enzymatic degradation of RDX. The product distribution of the present RDX hydrolysis is somewhat similar to that reported by Hoffsommer et al. (1977), Croce et al. (1979), and Heilmann et al. (1996). However, for the first time, 4-NDAB was detected as a key ring cleavage product in the hydrolysis of the three monocyclic nitramines (RDX, MNX and HMX) but not from the polycyclic one, CL-20 (Table 4 and FIG. 21).

In addition, it was found that MNX hydrolysis proceeds via initial denitration (i.e., elimination of HNO₂) followed by ring cleavage to produce NO₂⁻, N₂O (trace quantities), HCHO, and 4-NDAB (Table 4 and FIG. 21). Again, while we were unable to detect formamide, we did detect its hydrolyzed products HCOO⁻ (0.35 μmol) and NH3 (0.33 μmol). Likewise, HMX hydrolysis was accompanied by the release of 1 molar equivalent of NO₂⁻ with the formation of the end products N₂O, HCHO, and 4-NDAB (Table 4, FIG. 21 part C). Since the rate of HMX hydrolysis is more than an order of magnitude less than that of RDX hydrolysis, we did not follow the reaction to completion. Rather, we followed the reaction for only 21 days (ca. 5% of reaction). However, when we performed the reaction at pH 12.3, we detected NO₂⁻, N₂O, HCHO, 4-NDAB, NH3 and HCOO⁻ in less than 60 h. 4-NDAB was found to be unstable at pH 12.3 and decomposed to apparently produce NH₃ and HCOO⁻. Finally, when we hydrolyzed CL-20 at pH 10, 2 molar equivalents of NO₂⁻ were released instead of one and N₂O, NH₃, and HCOO⁻ accumulated. CL-20 hydrolysis occurred at a faster rate (1.09×10⁻² h⁻¹) than was observed for either RDX (7.21×10⁻³ h⁻¹) or HMX 1.02×10⁴ h⁻¹), but slower than we found for MNX (2.83×10⁻² h⁻¹). The faster rate of hydrolysis observed with CL-20 compared to RDX and HMX decomposition most likely arises from the highly strained nature of the polycyclic nitramine cage, which renders CL-20 more susceptible to nucleophilic attack.

We were unable to observe any of the RDX nitroso products (hexahydro-1-nitroso-3,5-dinitro-1,3,5-triazine (MNX), hexahydro-1,3-dinitroso-5-nitro-1,3,5-triazine (DNX), and hexahydro-1,3,5-trinitroso-1,3,5-triazine (TNX)), which is also consistent with other reports on the alkaline hydrolysis of RDX. Indeed we found that hydrolysis on MNX under the same conditions failed to produce the dinitroso (DNX) or the trinitroso (TNX) compounds. Likewise, when we hydrolyzed HMX and CL-20 at pH 10, none of their corresponding nitroso products were observed. These results provide strong evidence that initial denitration is a key step responsible for the decomposition of cyclic nitramines in water.

Table 4 illustrates normalized molar yields for the products obtained upon alkaline hydrolysis (pH 10) of the cyclic nitramines RDX, MNX, HMX, and CL-20. Stoichiometries are calculated based on the number of moles of product observed for each mole of reactant consumed.

TABLE 4
Normalized Molar Yields for Products of Alkaline
Hydrolysis of RDX, MNX, HMX, and CL-20

| | Observed Products^{d} | | | | |
|---|---|---|---|---|---|
| Compound | NO₂⁻ | N₂O | NH₄⁺ | 4-NDAB | HCHO |
| RDX^{a} | 0.972 ± 0.056 | 0.770 ± 0.130 | 0.075 ± 0.032 | 0.951 ± 0.005 | 0.939 ± 0.037 |
| MNX^{a} | 0.935 ± 0.005 | 0.008 ± 0.001 | 0.141 ± 0.035 | 0.842 ± 0.032 | 0.800 ± 0.027 |
| HMX^{b} | 1.15 ± 0.03 | 1.48 ± 0.06 | n.d.^{c} | 0.859 ± 0.016 | 1.82 ± 0.04 |
| CL-20^{b} | 1.91 ± 0.09 | 0.910 ± 0.100 | 0.793 ± 0.049 | not formed | not formed |

^{a}Values are the average of data from three replicate measurements.
^{b}Values are the average of date from duplicate measurements.
^{c}Due to its slow reaction rate, monitoring of HMX hydrolysis was stopped after 21 days. It is possible that NH₃ and HCOO⁻ formed, but were at levels less than the detection limit of the capillary electrophoresis method.
^{d}Values for HCOO⁻ are as follows for RDX^{a}, MNX^{a}, HMX^{b} and CL-20^{b} respectively: 0.070 ± 0.005; 0.152 ± 0.008;
n.d.^{c}; and 0.493 ± 0.007.

Table 4 summarizes the normalized molar yields for the products obtained in the hydrolysis of RDX, MNX, HMX and CL-20 at pH 10. From Table 4, it is apparent that, except for CL-20, these compounds lose one equivalent of NO₂⁻ and form HNNO₂CH₂NHCHO (4-NDAB) as a major product. For CL-20, we found that hydrolysis (pH 10) was accompanied by the release of two molar equivalents of nitrite ion rather than one and the formation of HCOOH rather than HCHO. The observation of NO₂⁻ emphasized once again the importance of initial denitration in the subsequent decomposition of CL-20. However, we were unable to observe 4-NDAB as was the case with the other nitramines examined. This is not surprising in view of the structural differences between the rigid caged polycyclic CL-20 which is characterized by the presence of C—C bonds (elongated) that are absent in the case of the mono cyclic ones (FIG. 1).

For RDX, it was found that C mass balance of 97% distributed as follows: HCHO (31.3%), HCOOH (2.3%), and HNNO₂CH₂NHCHO (63.4%) and a N mass balance of 90.7% distributed as follows: NO₂⁻ (16.2%), N₂O (25.6%), NH₃ measured as NH₄⁺ (1.3%), and HNNO₂CH₂NHCHO (47.6%). For MNX, it was found that C mass balance of 87.8% in: HCHO (26.6%), HCOOH (5.05%), and HNNO₂CH₂NHCHO (56.1%) and a N mass balance of 60.45% distributed as follows: NO₂⁻ (15.6%), N₂O (0.14%), NH₃ measured as NH₄⁺ (2.35%), and HNNO₂CH₂NHCHO (42.1%). Whereas for HMX, it was found that C mass balance of 90% distributed as follows: HCHO (46.2%), HNNO₂CH₂NHCHO (43.8%) and a N mass balance of 86% distributed as follows: NO₂⁻ (14.7%), N₂O (38.5%), HNNO₂CH₂NHCHO (32.8%). Due to the slow reaction rate of HMX at pH 10 the presence of NH₃ and HCOOH, could not be unequivocally confirmned. However, when the pH was increased to 12.3 a product distribution (NO₂⁻, N₂O, NH₃ and HCOOH) was obtained, but 4-NDAB was found to decompose at this pH thereby explaining why this important intermediate was previously found in hydrolysis studies.

Stoichiometry. Table 5 summarizes the percentage of N-containing and C-containing products produced by RDX degradation at pH 10. The stoichiometries of nitrogen and carbon during the alkaline hydrolysis of RDX is shown, calculated on the basis of percentage of reacted RDX using total theoretical number of N and C atoms. We found a C mass balance of 97% distributed as follows: HCHO (31.3%), HCHOO (2.3%), and V (63.4%) and a N mass balance of 90.7% distributed as follows: NO₂⁻ (16.2%), N₂O (25.6%), NH₃ measured as NH₄⁺ (1.3%), and V (47.6%).

TABLE 5
Stoichiometries of N and C During the alkaline hydrolysis of RDX^{a}

| Element | | | | | | | |
|---|---|---|---|---|---|---|---|
| (total no. | | | | | | | |
| of atoms) | NO₂⁻ | N₂O | NH₃ | CH₂O | CH₂OO | V | % Recovered |
| Carbon (3) | | | | 31.3 | 2.3 | 63.4 | 97 |
| Nitrogen | 16.2 | 25.6 | 1.3 | | | 47.6 | 93.5^{b} |
| (6) | | | | | | | |

^{a}Percentages are the average of data from three replicate measurements.
^{b}Total includes the amount of N₂O calculated to be in the aqueous phase, and also the amount calculated as being in the form of nitramide. Subtracting these theoretical values gives a total nitrogen recovery of 90.7%.

In addition, taking the concentrations of N₂O measured in these experiments as an equilibrium quantity, the aqueous phase concentration of N₂O can be calculated using Henry's Law. For N₂O, the Henry's Law co-efficient (kH) is 0.02522 M atm⁻¹ (Sander et al.). Furthermore, if it is assumed that the measured N₂O arose from the hydrolysis of nitramide (NH₂NO₂), the concentration of nitramide in solution can be calculated based on the rate (or half-life) of N₂O formation. With a half-life of 91 h, the reaction had proceeded to ca. 94% at the time the stoichiometry was quantified (t=316 h). Since the N₂O that was present accounted for 91% of the total nitrous oxide expected to form, it was assumed that the remainder was in the form of NH₂NO₂. Hence, 0.3% of the nitrogen is calculated as being in the form of aqueous N₂O, while 2.5% of the nitrogen is thought to be still in nitramide. Thus, the combination of observed and calculated nitrogen mass balances yields a total recovered nitrogen of 93.5%.

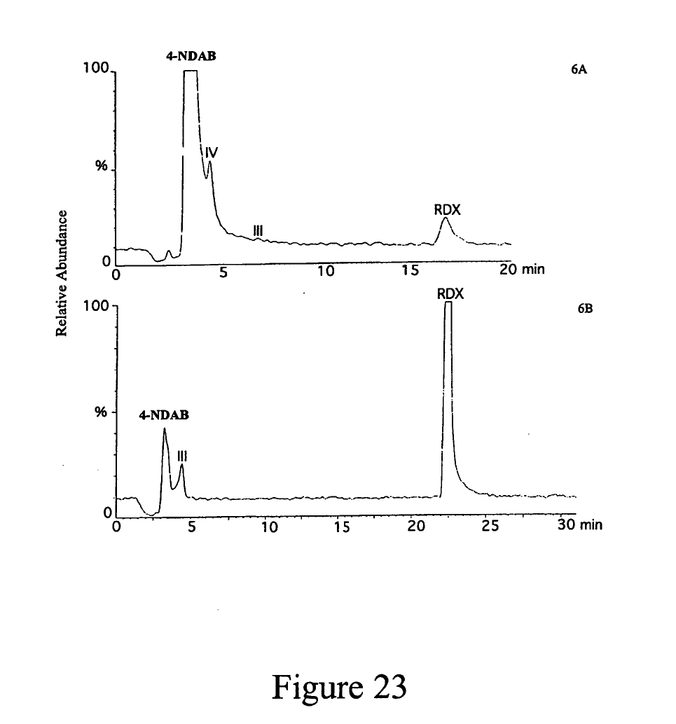

Identification of Early Intermediates in RDX Hydrolysis. In none of the LC/MS traces shown (FIG. 21A and FIG. 23) were we able to directly detect pentahydro-3,5-dinitro-1,3,5-triaza-1-ene (II). Since II is an enamine, it should react quite readily with water through its reactive —C═N bond, and in fact, it is known that II hydrolyzes at a rate 5 orders of magnitude higher than RDX (1). Therefore, we hydrolyzed RDX in a MeCN:H₂O mixture in the presence and absence of HP-β-CD in an attempt to generate and stabilize sufficient amounts of II and other suspected key intermediates to allow their detection.

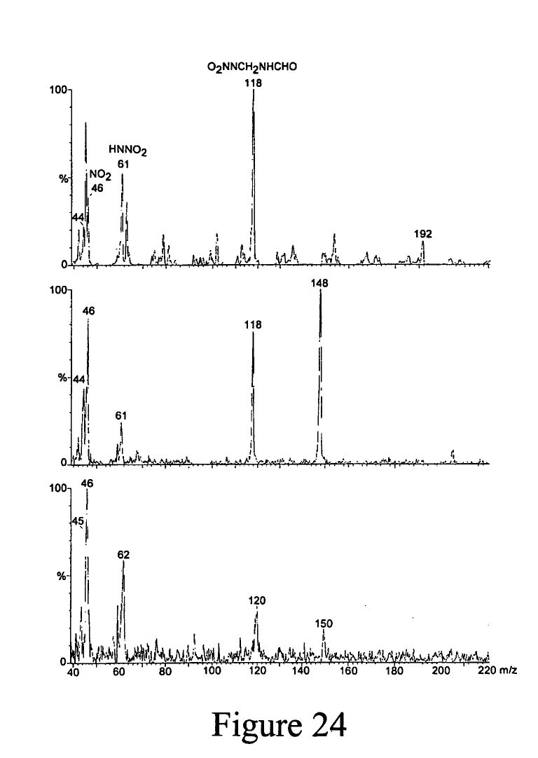

FIG. 23 part A shows that RDX hydrolysis in a MeCN:H₂O mixture (70% v/v) produced 4-NDAB (r.t 3.6 min) and two additional peaks marked III (r. t. 6.7 min) and IV (r.t. 4.4 min). III was detected only in trace amounts, but as discussed below, the presence of HP-β-CD increased its yield (FIG. 23 part B). FIG. 24 part A shows III with a [M−H] at 192 Da, matching a molecular mass formula of C₃H₇N₅O₅, and three other mass ion fragments at 46 Da, 61 Da and 118 Da, representing —NO₂, —NHNO₂, and the deprotonated mass ion of O₂NNHCH₂NHCHO (4-NDAB), respectively. When ring-labeled [¹⁵N]-RDX was used, the [M−H] ion appeared at 195 Da, confirming that this intermediate contained all three 15N atoms originally present in the ring structure of RDX. In addition, the mass ion fragments appearing at 118 and 61 Da were shifted to 120 Da (O₂¹⁴N¹⁵NHCH₂¹⁵NHCHO) and 62 Da (—15NH¹³NO₂), respectively. We tentatively identified III as the ring cleavage intermediate 4,6-dinitro-2,4,6-triaza-hexanal (O₂NNHCH₂NNO₂CH₂NHCHO), considered as a hydrolyzed product of the presumably formed denitrated cyclohexene intermediate (II) (FIG. 19). RDX photolysis in aqueous solution in a Rayonet photoreactor at 350 nm also produced intermediate O₂NNHCH₂NNO₂CH₂NHCHO, which was also attributed to the hydration of the initially formed denitrated intermediate II. This is the first report of the formation of intermediate O₂NNHCH₂NNO₂CH₂NHCHO via the alkaline hydrolysis of RDX.

Meanwhile, the peak labeled IV exhibited a [M−H] at 148 Da, matching a molecular mass formula of C₃H₇N₃O₄. IV showed three other mass ion fragments at 46, 61 and 118 Da, representing —NO₂, —NHNO₂, and the [M−H] of O₂NNHCH₂NHCHO, respectively (FIG. 24 part B). When ring-labeled [¹⁵N]-RDX was used, the [M−H] ion of IV shifted to 150 Da (FIG. 24 part C), indicating that the intermediate contained only two of the three ring-labeled ¹⁵N atoms in RDX. In addition, the [M−H] peaks that were detected at 118 and 61 Da shifted to 120 (O₂¹⁴N¹⁵NHCH₂¹⁵NHCHO) and 62 Da (—¹⁵NH¹⁴NO₂) Da, respectively. Therefore, the [M−H] peak detected at 148 Da probably arose from the ring cleavage product 5-hydroxy-4-nitro-2,4-diaza-pentanal (HOCH₂NNO₂CH₂NHCHO, IV) that formed upon hydrolysis of O₂NNHCH₂NNO₂CH₂NHCHO (III).

When RDX (1% w/v) was hydrolyzed in the presence of 3% HP-β-CD in a 65:35 (v/v) acetonitrile: water mixture at pH 12.3, we only observed III and 4-NDAB (FIG. 23 part B). As indicated above, we had hoped that the use of HP-β-CD during the hydrolysis of I would stabilize its initial denitrated intermediate (II) through the formation of an inclusion complex and thus facilitate its detection. Instead, it appears that the addition of HP-β-CD stabilized III, and allowed its unequivocal detection.

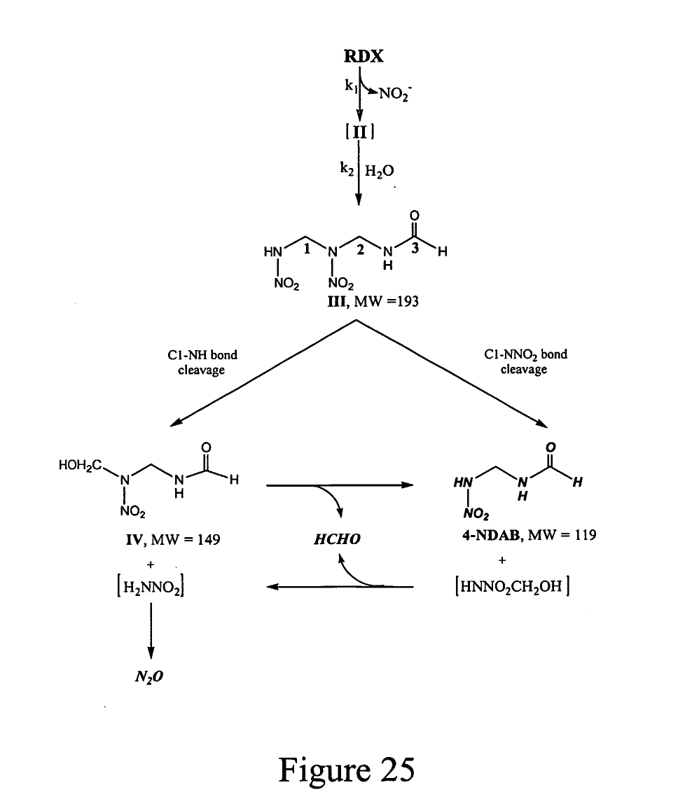

Decomposition Pathways. Based on product distribution and mass balance studies (Table 4 and Table 5) and the critical observation that denitration was a key step responsible for degradation of cyclic nitramines we proposed a degradation pathway that is consistent with previous reports (FIG. 19). However, the novel input in the present study is the discovery of several ring cleavage products including III, IV and 4-NDAB (FIG. 25). Although the initially denitrated intermediate 3,5-dinitro-1,3,5-triazacyclohex-1-ene (II) was not detected due to its rapid hydrolysis in water, its hydrolyzed product III was detected.

FIG. 20 shows that for every mole of RDX that disappears, approximately one mole of 4-nitro-2,4-diaza-butanal (4-NDAB) is formed. However, we found that the disappearance of 0.14 μmol of RDX was accompanied by the formation of only 0.04 pmol of 4-NDAB during the first three hours, suggesting that there are intermediates en route to 4-NDAB. Also, both intermediates III and IV were found to contain a strong mass ion fragment at 118 Da (FIG. 24) characteristic of the [M−H] assigned to 4-NDAB, suggesting that both III and IV act as precursors to the dead end product 4-NDAB.

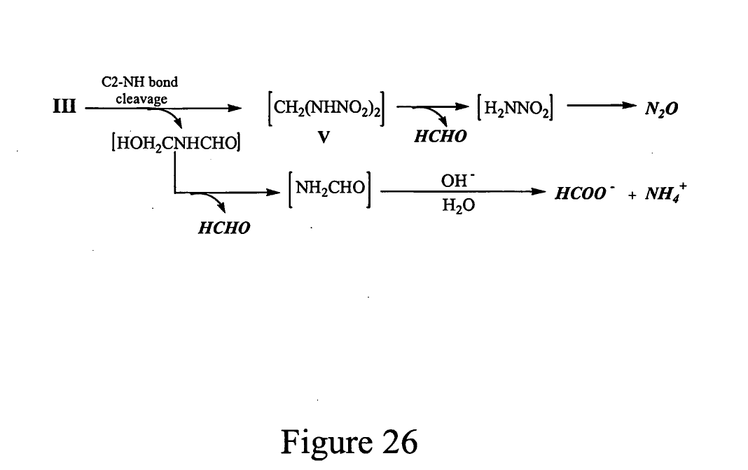

Intermediates III and IV have different C—N bonds that are subject to hydrolytic cleavage. A plausible route for the formation of the novel intermediate observed with a [M−H] at 148 Da (IV) could be via nucleophilic attack by OH at the C atom of the NH—Cl bond III, accompanied by the loss of NH₂NO₂ (FIG. 25). IV could then decompose to form 4-NDAB and HCHO. Alternatively, cleavage of the NNO₂—Cl bond in m would directly give rise to 4-NDAB and hydroxymethyl nitramide, O₂NNHCH₂OH (FIG. 25). However O₂NNHCH₂OH, being an a-hydroxyalkyl nitramine, is expected to be unstable in water and should thus decompose to HCHO and N₂O. Finally, cleavage at the C2-NH bond in III would lead to the production of methylene dinitramine (V) and formamide, NH₂CHO (FIG. 26). Although V was not detected, its presence cannot be excluded since it decomposes rapidly under alkaline conditions to produce HCHO and N₂O.

Clearly, nucleophilic attack at the C1 position in III is favoured because C1, being surrounded on both sides by stronger electron-withdrawing nitramine groups, is expected to be more electrophilic than C2. Consequently, C1 is more susceptible to nucleophilic attack by OH⁻ than C2, explaining why the route depicted in FIG. 26 (producing HCOO⁻ and NH₄⁺) is of only minor importance. Interestingly, III was observed during both RDX photo denitration at 350 nm and in the present Example of alkaline hydrolysis, yet IV was not observed prior to this work. However, the C1-NH bond cleavage that produces IV (FIG. 25) is favoured only when the solution pH exceeds the nitrarnine pKa of 6.55, at which point nitramine can serve as a leaving group. Hence, we were able to observe intermediate IV during alkaline hydrolysis at pH 10 but not during photolysis at pH 5.5.

Both MNX and HMX also hydrolyze via initial denitration to produce NO₂⁻, followed by a ring cleavage and spontaneous decomposition to N₂O, HCHO, and 4-NDAB. These findings indicate that the mechanism postulated for the alkaline hydrolysis of RDX applies more broadly to other mono cyclic nitramine explosives. For MNX, the analogous intermediate to III is ONNHC¹H₂NNO₂C²H₂NHCHO (4-nitro-6-nitroso-2,4,6-triaza-hexanal). Since the C-2 position of this analog is expected to be more electrophilic than the C-2 position in III itself, HNCHO (evolved as formamide) becomes more amenable for nucleophilic substitution by OH⁻. As a result, the minor pathway producing NH³ and HCOO⁻ (FIG. 26) is of somewhat more importance with MNX than it is for RDX (see Table 4 for product distribution).

In contrast to the single denitration event seen during either alkaline hydrolysis in this Example or photodenitration, two enzymatic denitration events occur en route to producing 4-NDAB. However, under extreme conditions ([NaOH]>19 M), two denitration events may also occur.

Although the polycyclic nitramine CL-20 is structurally different from the monocyclic RDX, MNX and HMX, its decomposition was also found to proceed via initial denitration (two molar equivalents of NO₂⁻) to give end products including N₂O, NH₃, and HCOO⁻ (Table 4).

In conclusion, the present Example confirms that a successful initial (primary) attack, denitration in this case, occurs in the degradation of monocyclic and polycyclic nitramines. Once denitration occurs, ring cleavage becomes spontaneous and is dictated by the chemistry of the ensuing intermediates in water. Similarities are observed between the decomposition patterns of cyclic nitramines under various chemical, biochemical and biological conditions. The rapid denitration process may be used as a probe to detect early stage decomposition events.

The above-described embodiments of the invention are intended to be examples of the present invention. Alterations, modifications and variations may be effected the particular embodiments by those of skill in the art, without departing from the scope of the invention which is defined solely by the claims appended hereto.

1. A process for degradation of cyclic nitramines comprising the steps of: attacking a first N—NO₂ group in the presence of water resulting in denitration of the first N—NO₂ group; and subsequently effecting ring cleavage.

2. A process according to claim 1 wherein a second N—NO₂ group is attacked after denitration of the first N—NO₂ group, resulting in a second denitration prior to effecting ring cleavage.

3. The process of claim 1 wherein the step of attacking a first N—NO₂ group comprises a chemical, biochemical or biological attack.

4. The process of claim 1 wherein the step of attacking a first N—NO₂ group comprises a chemical attack selected from the group consisting of photodenitration and alkaline hydrolysis.

5. The process of claim 1 wherein the step of attacking a first N—NO₂ group comprises a biochemical attack by exposure to an enzyme selected from the group consisting of diaphorase, cytochrome P450, xanthine oxidase, nitroreductase, and nitrate reductase.

6. The process of claim 1 wherein the step of attacking a first N—NO₂ group comprises a biological attack by exposure to a microbe selected from the group consisting of Clostridium kluyveri, Rhodococcus sp., and Aspergillus niger.

7. The process of claim 6 wherein Rhodococcus sp. comprises strain DN22.

8. The process of claim 1 wherein said nitramines comprise monocyclic or polycyclic nitramines.

9. The process of claim 1 wherein said nitramines are explosives selected from the group consisting of hexahydro-1,3,5-trinitro-1,3,5-triazine (RDX), HMX, CL20 and tetryl.

10. A process to detect decomposition of hexahydro-1,3,5-trinitro-1,3,5-triazine (RDX) comprising detection of 4-nitro-2,4-diaza-butanal (4-NDAB) as a decomposition product.

11. The process according to claim 4 wherein said chemical attack comprises photodenitration by photolysis at 350 nm in aqueous solution.

12. A process according to claim 1 wherein at least one step occurs in the presence of a cyclodextrin co-solvent.

13. A process for degradation of cyclic nitramines comprising the steps of: effecting α-hydroxylation of a —CH₂ bond to form unstable carbinol by exposure to cytochrome P450; and subsequently effecting ring cleavage.

14. A process for preventing sequential reduction of cyclic nitramine explosives to nitroso-derivatives comprising the steps of α-hydroxylation of a —CH₂ bond to form unstable carbinol; and subsequently effecting ring cleavage.

15. A process for degradation of 4-nitro-2,4-diazabutanal comprising exposure to an attack selected from the group consisting of anaerobic microbial attack, anaerobic enzymatic conditions, and chemical hydrolysis.

16. The process of claim 15 wherein said attack comprises an attack selected from the group consisting of exposure to one or more of diaphorase, cytochrome P450, xanthine oxidase and nitrate reductase; exposure to a microbe selected from the group consisting of Clostridium kluyveri, Rhodococcus sp., and Aspergillus niger; and alkaline hydrolysis.
